SEQUENCE LISTING (42 sequences)

SEQ ID NO: 1 - protein, 119 aa, synthetic construct
  REGION 1..119: note = Chemically Synthesized, hu1H7_VH v1
QVQLVQSGAEVKKPGASVKVSCKASGFTFTDYLIQWVRQAPGQGLEWIGWIYPGSGSIKYNEKFQGRVTMTADTSINTAYMELSRLRSDDTAVYFCARRGDWGGFFDYWGQGTLVTVSS

SEQ ID NO: 2 - protein, 119 aa, synthetic construct
  REGION 1..119: note = Chemically Synthesized, hu1H7_VH v2
QVQLVQSGAEVKKPGASVKLSCKASGFTFTDYLIQWVRQAPGQGLEWIGWFYPGSGSIKYNEKFKDRATLTADKSINTVYMELSRLRSDDTAVYFCARRGDWGGFFDYWGQGTLVTVSS

SEQ ID NO: 3 - protein, 119 aa, synthetic construct
  REGION 1..119: note = Chemically Synthesized, 1H7_VH
QVQLQQSGAELVKPGASVKLSCKASGFTFTDYLIQWVKQRSGQGLEWIGWFYPGSGSIKYNEKFKDKATLTADKSSNTVYMEISRLTSEDSAVYFCARRGDWGGFFDYWGQGTTLTVSS

SEQ ID NO: 4 - protein, 98 aa, synthetic construct
  REGION 1..98: note = Chemically Synthesized, IGHV1-2*02F
QVQLVQSGAEVKKPGASVKVSCKASGYTFTGYYMHWVRQAPGQGLEWMGWINPNSGGTNYAQKFQGRVTMTRDRSISTAYMELSRLRSDDTAVYYCAR

SEQ ID NO: 5 - protein, 10 aa, synthetic construct
  REGION 1..10: note = Chemically Synthesized, Hu1H7, Heavy Chain CDR1
GFTFTDYLIQ

SEQ ID NO: 6 - protein, 20 aa, synthetic construct
  REGION 1..20: note = Chemically Synthesized, Hu1H7, Heavy Chain CDR2
WIGWIYPGSGSIKYNEKFQG

SEQ ID NO: 7 - protein, 12 aa, synthetic construct
  REGION 1..12: note = Chemically Synthesized, Hu1H7, Heavy Chain CDR3
ARRGDWGGFFDY

SEQ ID NO: 8 - protein, 107 aa, synthetic construct
  REGION 1..107: note = Chemically Synthesized, hu1H7_VL v1
EIVLTQSPATLSLSPGERATLSCTASSSVSSSYLHWYQQKPGLAPRLWIFSTSNLASGIPDRFSGSGSGTDFTLTISRLEPEDFAVYYCHQYHRSLTFGGGTKVEIK

SEQ ID NO: 9 - protein, 107 aa, synthetic construct
  REGION 1..107: note = Chemically Synthesized, hu1H7_VL v2
DIVLTQSPATLSLSPGERATLSCTASSSVSSSYLHWYQQKPGSAPRLWIFSTSNLASGIPDRFSGGSGSTDYTLTISRLEPEDFAVYYCHQYHRSLTFGGGTKVEIK

SEQ ID NO: 10 - protein, 107 aa, synthetic construct
  REGION 1..107: note = Chemically Synthesized, 1H7_VL
QIVLTQSPAIMSASLGERVTMTCTASSSVSSSYLHWYQQKPGSSPKLWIFSTSNLASGVPDRFSGSGSGTSYSLTINSMEAEDAATYYCHQYHRSLTFGAGTKLELK

SEQ ID NO: 11 - protein, 96 aa, synthetic construct
  REGION 1..96: note = Chemically Synthesized, IGKV3D-20 (A11)
EIVLTQSPATLSLSPGERATLSCGASQSVSSSYLAWYAAKPGLAPRLLIYDASSRATGIPDRFSGSGSGTDFTLTISRLEPEDFAVYYCQQYGSSP

SEQ ID NO: 12 - protein, 14 aa, synthetic construct
  REGION 1..14: note = Chemically Synthesized, Hu1H7, Light Chain CDR1
TASSSVSSSYLHWY

SEQ ID NO: 13 - protein, 11 aa, synthetic construct
  REGION 1..11: note = Chemically Synthesized, Hu1H7, Light Chain CDR2
LWIFSTSNLAS

SEQ ID NO: 14 - protein, 8 aa, synthetic construct
  REGION 1..8: note = Chemically Synthesized, Hu1H7, Light Chain CDR3
HQYHRSLT

SEQ ID NO: 15 - protein, 118 aa, synthetic construct
  REGION 1..118: note = Chemically Synthesized, hu2A9 VH
QVQLVQSGAEVKKPGASVKLSCKASGYTFTSYWMQWVRQAPGQGLEWIGAIYPGDGETTYTQKFKGRVTMTADTSISTAYMELSRLRSDDTAVYYCAKGDGYWAMDYWGQGTLVTVSS

SEQ ID NO: 16 - protein, 98 aa, synthetic construct
  REGION 1..98: note = Chemically Synthesized, IGHV1-2*02F
QVQLVQSGAEVKKPGASVKVSCKASGYTFYGYYMHWVRQAPGQGLEWMGWINPNSGGTNYAQKFQGRVTMTRDTSISTAYMELSRLRSDDTAVYYCAR

SEQ ID NO: 17 - protein, 118 aa, synthetic construct
  REGION 1..118: note = Chemically Synthesized, 2A9_VH
QVQLQQSGAELARPGASVKLSCKASGYTFTSYWMQWVKQRPGQGLEWIGAIYPGDGETTYTQKFKGKATLTADKSSSTAYMQLSSLASEDSAVYYCAKGDGYWAMDYWGQGTSVTVSS

SEQ ID NO: 18 - protein, 10 aa, synthetic construct
  REGION 1..10: note = Chemically Synthesized, Hu2A9, Heavy Chain CDR1
GYTFTSYWMQ

SEQ ID NO: 19 - protein, 20 aa, synthetic construct
  REGION 1..20: note = Chemically Synthesized, Hu2A9, Heavy Chain CDR2
WIGAIYPGDGETTYTQKFKG

SEQ ID NO: 20 - protein, 11 aa, synthetic construct
  REGION 1..11: note = Chemically Synthesized, Hu2A9, Heavy Chain CDR3
AKGDGYWAMDY

SEQ ID NO: 21 - protein, 112 aa, synthetic construct
  REGION 1..112: note = Chemically Synthesized, hu2A9 VL
DVVMTQSPLSLPVTLGQPASISCRSSQSLVHSNGNTYLHWYQQRPGQSPRLLIYKVSNRFSGVPDRFSGSGSGTDFTLKISRVEAEDVGVYYCSQSTHVPLTFGAGTKLELK

SEQ ID NO: 22 - protein, 100 aa, synthetic construct
  REGION 1..100: note = Chemically Synthesized, IGKV2-30*02
DVVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWP

SEQ ID NO: 23 - protein, 112 aa, synthetic construct
  REGION 1..112: note = Chemically Synthesized, 2A9_VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 24 - protein, 18 aa, synthetic construct
  REGION 1..18: note = Chemically Synthesized, Hu2A9, Light Chain CDR1
RSSQSLVHSNGNTYLHWY

SEQ ID NO: 25 - protein, 11 aa, synthetic construct
  REGION 1..11: note = Chemically Synthesized, Hu2A9, Light Chain CDR2
LLIYKVSNRFS

SEQ ID NO: 26 - protein, 9 aa, synthetic construct
  REGION 1..9: note = Chemically Synthesized, Hu2A9, Light Chain CDR3
SQSTHVPLT

SEQ ID NO: 27 - DNA, 30 bp, synthetic construct
  misc_feature 1..30: note = Chemically Synthesized
  source 1..30: mol_type = other DNA
ggcttcaccttcaccgactacctgatccag

SEQ ID NO: 28 - DNA, 60 bp, synthetic construct
  misc_feature 1..60: note = Chemically Synthesized
  source 1..60: mol_type = other DNA
tggatcggctggatctaccctggcagcggcagcatcaagtacaacgagaagttccagggc

SEQ ID NO: 29 - DNA, 36 bp, synthetic construct
  misc_feature 1..36: note = Chemically Synthesized
  source 1..36: mol_type = other DNA
gccagaagaggcgactggggcggcttcttcgattac

SEQ ID NO: 30 - DNA, 42 bp, synthetic construct
  misc_feature 1..42: note = Chemically Synthesized
  source 1..42: mol_type = other DNA
accgctagcagcagcgtgtccagcagctacctgcactggtat

SEQ ID NO: 31 - DNA, 33 bp, synthetic construct
  misc_feature 1..33: note = Chemically Synthesized
  source 1..33: mol_type = other DNA
ctgtggatcttcagcaccagcaatctggcctcc

SEQ ID NO: 32 - DNA, 24 bp, synthetic construct
  misc_feature 1..24: note = Chemically Synthesized
  source 1..24: mol_type = other DNA
caccagtaccacagaagcctgacc

SEQ ID NO: 33 - DNA, 30 bp, synthetic construct
  misc_feature 1..30: note = Chemically Synthesized
  source 1..30: mol_type = other DNA
ggctacaccttcaccagctactggatgcag

SEQ ID NO: 34 - DNA, 60 bp, synthetic construct
  misc_feature 1..60: note = Chemically Synthesized
  source 1..60: mol_type = other DNA
tggatcggcgccatctatcctggcgacggcgagacaacctacacccagaaattcaagggc

SEQ ID NO: 35 - DNA, 33 bp, synthetic construct
  misc_feature 1..33: note = Chemically Synthesized
  source 1..33: mol_type = other DNA
gccaagggcgacggctactgggctatggattat

SEQ ID NO: 36 - DNA, 54 bp, synthetic construct
  misc_feature 1..54: note = Chemically Synthesized
  source 1..54: mol_type = other DNA
cggagcagccagagcctggtgcacagcaacggcaacacctacctgcactggtat

SEQ ID NO: 37 - DNA, 33 bp, synthetic construct
  misc_feature 1..33: note = Chemically Synthesized
  source 1..33: mol_type = other DNA
ctgctgatctacaaggtgtccaacagattcagc

SEQ ID NO: 38 - DNA, 27 bp, synthetic construct
  misc_feature 1..27: note = Chemically Synthesized
  source 1..27: mol_type = other DNA
tcccagagcacccacgtgcccctgacc

SEQ ID NO: 39 - protein, 15 aa, synthetic construct
  REGION 1..15: note = Chemically Synthesized, JH4
YFDYWGQGTLVTVSS

SEQ ID NO: 40 - protein, 12 aa, synthetic construct
  REGION 1..12: note = Chemically Synthesized, IGKJ4
LTFGGGTKVEIK

SEQ ID NO: 41 - protein, 15 aa, synthetic construct
  REGION 1..15: note = Chemically Synthesized, JH4
YFDYWGQGTLVTVSS

SEQ ID NO: 42 - protein, 12 aa, synthetic construct
  REGION 1..12: note = Chemically Synthesized, IGKJ4
LTFGGGTKVEIK

(19) United States
(12) Patent Application Publication
(10) Pub. No.: US 2024/0026025 A1
(43) Pub. Date: Jan. 25, 2024

(54) HUMANIZED ANTI-N-CADHERIN ANTIBODIES AND USES THEREOF

(71) Applicant: THE REGENTS OF THE UNIVERSITY OF CALIFORNIA, Oakland, CA (US)

(72) Inventors: Kirstin A. Zettlitz, Sherman Oaks, CA (US); Anna M. Wu, Sherman Oaks, CA (US); Robert E. Reiter, Los Angeles, CA (US)

(21) Appl. No.: 18/331,623

(22) Filed: Jun. 8, 2023

Related U.S. Application Data
(63) Continuation of application No. 16/619,496, filed on Dec. 5, 2019, now abandoned.
(60) Provisional application No. 62/515,617, filed on Jun. 6, 2017.

(51) Int. Cl. C07K 16/28; A61P 35/00

(52) U.S. Cl. CPC C07K 16/2896; A61P 35/00; A61K 38/00

(57) ABSTRACT

This invention relates to inhibition of the N-cadherin signaling using a humanized anti-N-cadherin antibody. In one embodiment, the invention relates to methods of treating an N-cadherin-mediated disease or N-cadherin-mediated disorder in a subject by contacting the subject with an anti-N-cadherin antibody.

### CROSS-REFERENCE TO RELATED APPLICATION

This application claims priority to U.S. Provisional Application Ser. No. 62/515,617, filed Jun. 6, 2017, the content of which is incorporated by reference herein in its entirety.

### STATEMENT REGARDING FEDERALLY SPONSORED RESEARCH OR DEVELOPMENT

This invention was made with Government support under CA092131, awarded by the National Institutes of Health. The Government has certain rights in the invention.

### BACKGROUND OF THE INVENTION

N-cadherin (Cadherin-2, CDH2) is a transmembrane protein involved in cell-cell adhesion that is expressed in multiple tissues. N-cadherin is a key marker of epithelial-to-mesenchymal transition (EMT), a process that plays a pivotal role in cancer progression. EMT and the associated N-cadherin expression promote invasion and metastasis in multiple malignancies, lead to treatment resistance to targeted therapies, and cause the rise of cancer stem cells. Consequently, N-cadherin provides an attractive target for diagnosis, therapy, and monitoring of disease progression.

EMT in cancer progression may be highly context-specific, but with regard to prostate cancer, EMT is induced following castration or by androgen deprivation therapy (ADT). The up-regulation of N-cadherin and concurrent repression of E-cadherin are thought to be involved in drug resistance, initiation of metastases and given rise to cancer stem cells (Nouri et al. 2014).

N-cadherin consists of five extracellular cadherin repeats, a transmembrane region and a highly conserved cytoplasmic tail. While the cytoplasmic tail mediates binding to catenins, which in turn interact with the actin cytoskeleton, the extracellular domains mediate homophilic interactions between adjacent cells. Murine monoclonal antibodies targeting N-cadherin domains 2 (1H7), and ¾ (2A9), respectively, have shown inhibitory effects on multiple prostate cancer models in vivo (Tanaka et al. 2010).

Thus, there is a need in the art for humanized antibodies that recognize human N-cadherin that can provide improved treatments for diseases associated with expression of N-cadherin. The present invention addresses and meets these and other needs.

### SUMMARY OF THE INVENTION

In one embodiment, the present invention relates to a humanized N-cadherin antibody that specifically binds to the extracellular domain of human N-cadherin. In some embodiments, the antibody includes at least one CDR comprising a sequence that is at least 90% identical to a sequence selected from the group consisting of: VH-CDR1 (SEQ ID NO:5); VH-CDR2 (SEQ ID NO:6); VH-CDR3 (SEQ ID NO:7); VL-CDR1 (SEQ ID NO:12); VL-CDR2 (SEQ ID NO:13); and VL-CDR3 (SEQ ID NO:14). In some embodiments, the antibody includes the CDRs having a sequence that is at least 90% identical to: VH-CDR1 (SEQ ID NO:5); VH-CDR2 (SEQ ID NO:6); VH-CDR3 (SEQ ID NO:7); VL-CDR1 (SEQ ID NO:12); VL-CDR2 (SEQ ID NO:13); and VL-CDR3 (SEQ ID NO:14).

In some embodiments, the antibody has a heavy chain that is at least 90% identical to the amino acid sequence of SEQ ID NO: 1. In some embodiments, the antibody has a light chain that is at least 90% identical to the amino acid sequence of SEQ ID NO:8. In some embodiments, the antibody includes a heavy chain that is at least 90% identical to the amino acid sequence of SEQ ID NO:1 and a light chain that is at least 90% identical to the amino acid sequence of SEQ ID NO:8. In some embodiments, the antibody includes at least one of the CDRs has a sequence that is at least 90% identical to a sequence selected from the group consisting of: VH-CDR1 (SEQ ID NO:18); VH-CDR2 (SEQ ID NO:19); VH-CDR3 (SEQ ID NO:20); VL-CDR1 (SEQ ID NO:24); VL-CDR2 (SEQ ID NO:25); and VL-CDR3 (SEQ ID NO:26). In some embodiments, the antibody comprises the CDRs comprising a sequence that is at least 90% identical to: VH-CDR1 (SEQ ID NO:18); VH-CDR2 (SEQ ID NO:19); VH-CDR3 (SEQ ID NO:20); VL-CDR1 (SEQ ID NO:24); VL-CDR2 (SEQ ID NO:25); and VL-CDR3 (SEQ ID NO:26).

In some embodiments, the antibody includes a heavy chain that is at least 90% identical to the amino acid sequence of SEQ ID NO:15. In some embodiments, the antibody includes a light chain that is at least 90% identical to the amino acid sequence of SEQ ID NO:21. In some embodiments, the antibody includes a heavy chain that is at least 90% identical to the amino acid sequence of SEQ ID NO:15 and a light chain that is at least 90% identical to the amino acid sequence of SEQ ID NO:21. In some embodiments, the antibody includes an effector moiety, wherein the effector moiety is selected from the group consisting of a radioactive label, a fluorescent label, or a therapeutic moiety.

In one aspect, the present invention provides a method of treating a N-cadherin-mediated disease or disorder in a subject, including the step of administering to the subject an effective amount of any antibody described herein, wherein when the antibody is administered, the disease or disorder is reduced. In some embodiments, the disease or disorder is at least selected from the group consisting of prostate cancer, bladder cancer, hormone refractory disease, carcinoma, melanoma, breast cancer, adrenal tumors, or any combinations thereof. In some embodiments, administration of the anti-N-cadherin antibody inhibits the function of N-cadherin protein.

In one aspect, the present invention provides a method of reducing the activity of N-cadherin of a subject, wherein the method comprises administering an effective amount of an antibody to the subject, wherein the antibody comprises any antibody described herein.

In one aspect, the present invention provides a method of detecting an N-cadherin-mediated disease or disorder in a subject, the method comprising administering to the subject any antibody described herein, detecting the level of the effector moiety, and measuring the level of the effector moiety relative to a comparator control. In some embodiments, the N-cadherin-mediate disease or disorder is selected from the group consisting of prostate cancer, bladder cancer, hormone refractory disease, or any combinations thereof.

In some embodiments, the present invention provides a method of reducing the activity of N-cadherin of a subject, wherein the method comprises administering an antibody to the subject wherein the antibody comprises any antibody described herein.

### BRIEF DESCRIPTION OF THE DRAWINGS

The foregoing summary, as well as the following detailed description of preferred embodiments of the invention, will be better understood when read in conjunction with the appended drawings. It should be understood, however, that the invention is not limited to the precise arrangements and instrumentalities of the embodiments shown in the drawings. In the drawings:

FIG. 1 depicts the sequence alignment of VH 1H7. Alignment of the VH sequence isolated from hybridoma 1H7 with the closest human germline sequence used as scaffold for humanization and both versions of the humanized sequence are shown. Sequence similarity is indicated by black (100%), grey (75%) or white (50% or less) background. The indicated CDR regions include Chothia, ABM, Kabat and contact definition.

FIG. 2 depicts the sequence alignment of VL of 1H7. Alignment of the VL sequence isolated from hybridoma 1H7 with the closest human germline sequence used as scaffold for humanization and both versions of the humanized sequence are shown.

FIG. 3 depicts the sequence alignment of VH of 2A9. Alignment of the VH sequence isolated from hybridoma 2A9 with the closest human germline sequence used as scaffold for humanization and the humanized sequence is shown.

FIG. 4 depicts sequence alignment of VI of 2A9. Alignment of the VL sequence isolated from hybridoma 2A9 with the closest human germline sequence used as scaffold for humanization and the humanized sequence is shown.

FIG. 5 depicts the superimposed model structure of Fv mo2A9 (blue) and Fv hu2A9 (purple). A side view of the two model structures is shown. The lighter regions show the grafted CDR regions. Models were generated with WAM and visualized with PyMOL (The PyMOL Molecular Graphics System, Version 1.8 Schrödinger, LLC.).

FIG. 6, comprising FIG. 6A and FIG. 6B, depicts purified humanized scFv hu2A9 and hu1H7. FIG. 6A illustrates hu2A9 scFv SDS-PAGE analysis (2 μg/lane, reducing and non-reducing conditions) and Western Blot (1 μg/lane). FIG. 6B depicts Hu1H7 scFv version 1 and 2 (v1, v2) SDS-PAGE analysis (1 μg/lane non-reducing conditions) and Western Blot (1 μg/lane). SDS-gels were stained with InstantBlue, Western blots were detected with HisDetector Western Blot Kit (KPL Cat. No. 25-00-01).

FIG. 7, comprising FIG. 7A through FIG. 7D, depicts immunoblot analysis of antigen binding and epitope mapping. Recombinant human ECD-Fc fusion proteins and commercial human CDH2 were blotted (0.5 μg/lane) and detected using anti-mouse Fcg-AP (JIR 115-055-071) and His Detector Western Blot Kit (FIG. 7A); mo2A9 scFv (FIG. 7B); hu2A9 scFv (FIG. 7C) and hu1H7 scFv v1 (FIG. 7D). Bound scFv fragments were detected using anti-c-Myc and anti-rabbit-AP antibodies as described above.

FIG. 8 depicts results from experiments evaluating the binding of humanized anti-N-cadherin scFv fragments to recombinant human N-cadherin ECD1-3-Fc in ELISA. Bound scFv was detected using Anti-c-Myc polyclonal rabbit (Sigma, C3956) and Anti-Rabbit (H+L)-AP (Jackson ImmunoResearch 111-055-144) antibodies.

FIG. 9, comprising FIG. 9A and FIG. 9B, depicts SDS-PAGE (FIG. 9A) and Western blot (FIG. 9B) analysis of hu2A9 IgG. Lane M: Protein Marker; Lane 1: Reducing conditions; Lane 2: Non-reducing conditions; Lane P: Human IgG1, Kappa (Sigma, Cat. No. I5154), positive control. Western Blot was detected using goat anti-human IgG-HRP (GenScript, Cat. No. A00166).

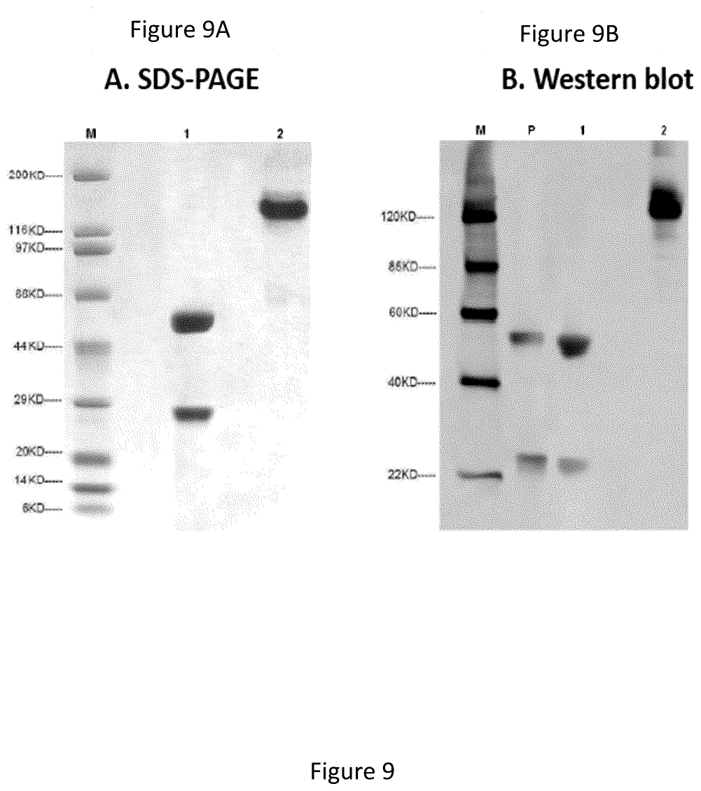

FIG. 10, comprising FIG. 10A and FIG. 10B, depicts SDS-PAGE (FIG. 10A) and Western blot (FIG. 10B) analysis of hu1H7 IgG. Lane M: Protein Marker; Lane 1: Reducing conditions; Lane 2: Non-reducing conditions; Lane P: Human IgG1, Kappa (Sigma, Cat. No. I5154), positive control. Western Blot was detected using goat anti-human IgG-HRP (GenScript, Cat. No. A00166).

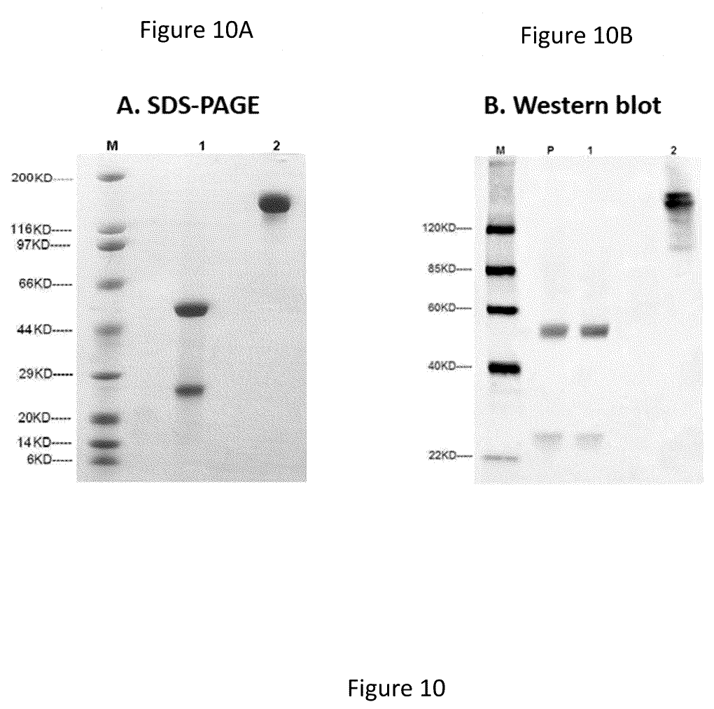

FIG. 11 depicts saturation binding of full length IgGs to recombinant antigen in ELISA. Bacterially produced antigens ECD4 and ECD1-3 were immobilized. Bound antibodies were detected with goat anti-human Fcg-AP or goat anti-mouse Fcg-AP respectively.

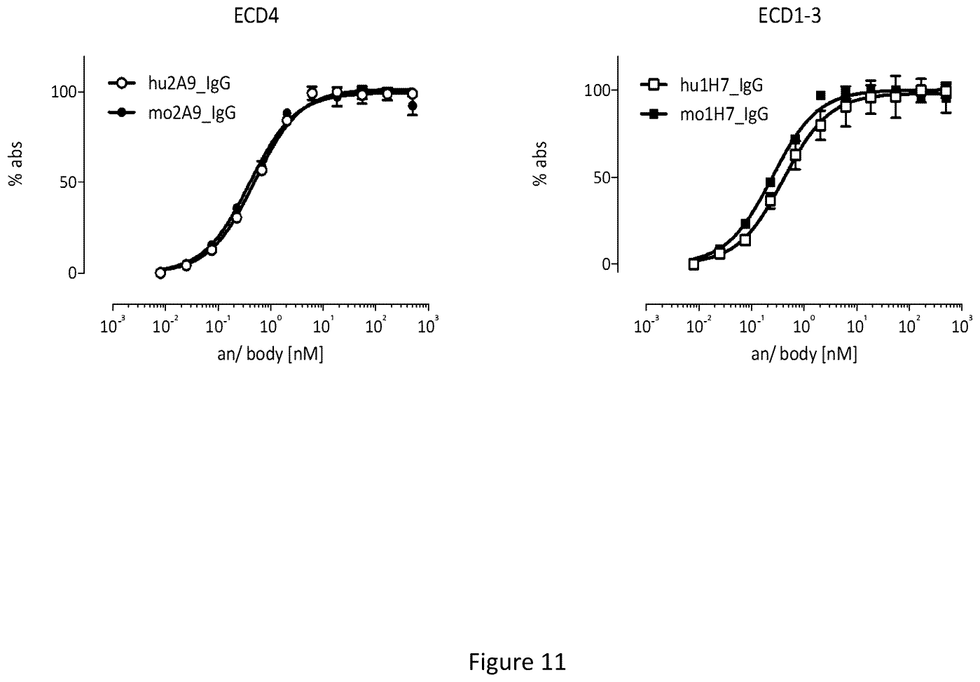

FIG. 12 depicts results demonstrating that anti-N-cadherin antibodies inhibit growth of s.c. xenografts (LNCaP-C1) in SCID mice. Subcutaneous tumors were treated upon reaching 100 mm³ with antibody (10 mg/kg, 3/weeks, i.p.). Tumor volumes were caliper measured.

FIG. 13 illustrates an additive effect of anti-N-cadherin antibodies and enzalutamide. Subcutaneous LNCaP-N tumors in SCID mice (n=9 per group) were treated with 10 mg/kg enzalutamide, 3-5 times per week.

FIG. 14 depicts the peptide sequence of the variable heavy chain and variable light chain of the human 1H7 antibody.

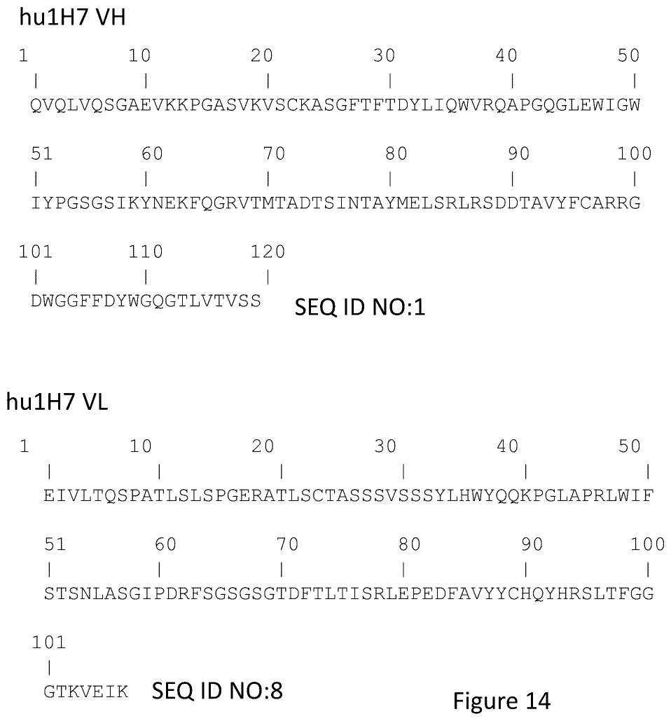

FIG. 15 depicts the peptide sequence of the variable heavy chain and variable light chain of the human 2A9 antibody.

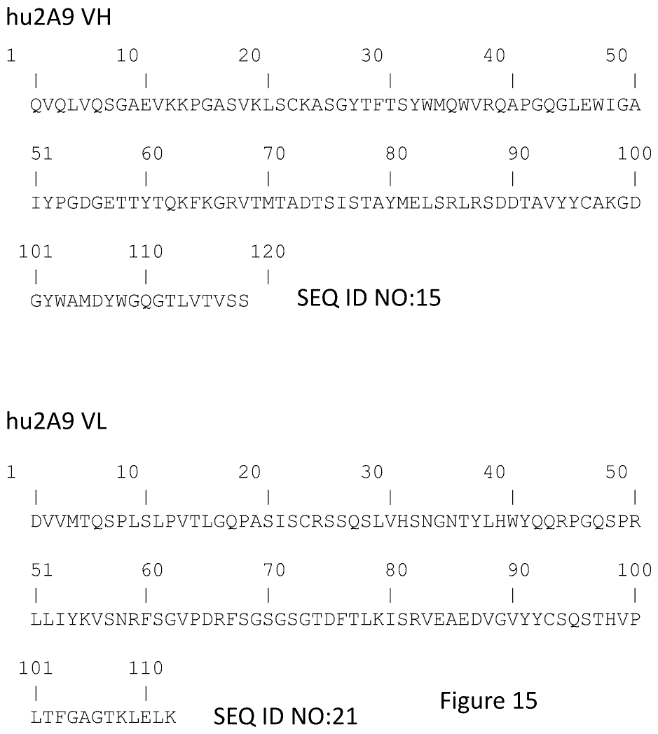

### DETAILED DESCRIPTION OF THE INVENTION

The present invention relates to compositions and methods for treating cancer. In some embodiments, the invention relates to therapeutic humanized antibodies recognizing human N-cadherin for the diagnosis and treatment of multiple cancers including but not limited to prostate cancer, hormone-refractory prostate cancer, bladder cancer carcinoma, melanoma, breast cancer, adrenal tumors, and the like.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The terms “inhibit” and “inhibition,” as used herein, means to reduce, suppress, diminish or block an activity or function by at least about 10% relative to a control value. Preferably, the activity is suppressed or blocked by 50% compared to a control value, more preferably by 75%, and even more preferably by 95%.

The terms “effective amount” and “pharmaceutically effective amount” refer to a sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

The terms “patient,” “subject,” “individual,” and the like are used interchangeably herein, and refer to any animal, in some embodiments a mammal, and in some embodiments a human, including a human in need of therapy for, or susceptible to, a condition or its sequelae. The individual may include, for example, dogs, cats, pigs, cows, sheep, goats, horses, rats, monkeys, and mice and humans.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected/homeostatic) respective characteristic. Characteristics which are normal or expected for one cell, tissue type, or subject, might be abnormal for a different cell or tissue type.

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

“Operably linked” or “operatively linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “therapeutic treatment” is a treatment administered to a subject who exhibits signs of disease or disorder, for the purpose of diminishing or eliminating those signs.

As used herein, “treating a disease or disorder” means reducing the frequency and/or severity of a sign and/or symptom of the disease or disorder is experienced by a patient.

The phrase “biological sample”, “sample” or “specimen” as used herein, is intended to include any sample comprising a cell, a tissue, or a bodily fluid in which expression of a nucleic acid or polypeptide can be detected. The biological sample may contain any biological material suitable for detecting the desired biomarkers, and may comprise cellular and/or non-cellular material obtained from the individual. Examples of such biological samples include but are not limited to blood, lymph, bone marrow, biopsies and smears. Samples that are liquid in nature are referred to herein as “bodily fluids.” Biological samples may be obtained from a patient by a variety of techniques including, for example, by scraping or swabbing an area or by using a needle to obtain bodily fluids. Methods for collecting various body samples are well known in the art.

The term “antibody,” as used herein, refers to an immunoglobulin molecule which is able to specifically bind to a specific epitope of an antigen. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from a subject with heavy chain disease, or prepared by the cloning and expression of VH (variable heavy chain immunoglobulin) genes from a subject.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., 1989, Queen et al., Proc. Natl. Acad Sci USA, 86:10029-10032; 1991, Hodgson et al., Bio/Technology, 9:421). A suitable human acceptor antibody may be one selected from a conventional database, e.g., the KABAT database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies (see for example EP-A-0239400 and EP-A-054951).

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but in some embodiments all) of the amino acid sequences encoding its heavy and/or light chain framework regions and/or its heavy and/or light chain constant regions to the first immunoglobulin partner. In certain embodiments a human antibody is the acceptor antibody.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p 877-883.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding”, can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Differentially decreased expression” or “down regulation” refers to biomarker product levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a control.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a control.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “DNA” as used herein is defined as deoxyribonucleic acid.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein refers to the specific group of atoms on an antigen molecule to which a specific antibody binds, causing an immune response.

The term “acceptor” as used herein refers to a molecule that provides the structural framework for creation of a humanized molecule, such as a human immunoglobulin.

The term “donor” as used herein refers to the molecule that provides the binding site element of a humanized molecule. This molecule is generally a non-human polypeptide, such as a murine monoclonal antibody.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Isolated” means altered or removed from the natural state. For example, a nucleic acid or a peptide naturally present in its normal context in a living subject is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

The phrase “humanized antibody” as used herein refers to a genetically-engineered antibody wherein the variable region comprises the CDRs or portions of the CDRs of a non-human antibody and the framework regions of a human antibody, and the constant region comprises the constant region of a human antibody.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “progeny” as used herein refers to a descendent or offspring and includes the offspring of a mammal, and also included the differentiated or undifferentiated decedent cell derived from a parent cell. In one usage, the term progeny refers to a descendent cell which is genetically identical to the parent. In another use, the term progeny refers to a descendent cell which is genetically and phenotypically identical to the parent. In yet another usage, the term progeny refers to a descendent cell that has differentiated from the parent cell.

The term “RNA” as used herein is defined as ribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

The term “regulating” as used herein can mean any method of altering the level or activity of a substrate. Non-limiting examples of regulating with regard to a protein include affecting expression (including transcription and/or translation), affecting folding, affecting degradation or protein turnover, and affecting localization of a protein. Non-limiting examples of regulating with regard to an enzyme further include affecting the enzymatic activity. “Regulator” refers to a molecule whose activity includes affecting the level or activity of a substrate. A regulator can be direct or indirect. A regulator can function to activate or inhibit or otherwise modulate its substrate.

A “scanning window”, as used herein, refers to a segment of a number of contiguous positions in which a sequence may be evaluated independently of any flanking sequence. A scanning window generally is shifted incrementally along the length of a sequence to be evaluated with each new segment being independently evaluated. An incremental shift may be of 1 or more than one position.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

This invention relates to a composition directed to the inhibition of N-cadherin, N-cadherin-related signaling, and N-cadherin-related disorders using a humanized anti-N-cadherin antibody. In one embodiment, the invention is directed to inhibiting N-cadherin signaling by specifically targeting the extracellular domain of N-cadherin protein. In one embodiment, the invention is directed to methods of treating and preventing diseases mediated by unwanted, uncontrolled, excessive N-cadherin expression or activity. In one embodiment, the invention is directed towards the treatment of N-cadherin-mediated disease or N-cadherin-mediated disorder in an individual by contacting the individual with an anti-N-cadherin antibody.

In one aspect, the invention relates to the inhibition of epithelial to mesenchymal transition (EMT), which is one type of cell movement than can be observed in embryogenesis requires the loss of cell-cell contacts for the migration of individual cells or small group of cells through the extracellular matrix. EMT also occurs in pathological situations, such as the acquisition of a motile and invasive phenotype of tumor cells of epithelial origin. A hallmark of EMT, is the loss of E-cadherin and the de novo expression of N-cadherin adhesion molecules. N-cadherin promotes tumor cell survival, migration and invasion, and high levels of N-cadherin expression is often associated with poor prognosis. N-cadherin is also expressed in endothelial cells and plays an essential role in the maturation and stabilization of normal vessels and tumor-associated angiogenic vessels. Increasing experimental evidence suggests that N-cadherin is a potential therapeutic target in cancer.

In one embodiment, the composition comprises a humanized anti-N-cadherin antibody. In some embodiments, the composition comprises a humanized anti-N-cadherin antibody and therapeutic agent combination. In some embodiments, the therapeutic agent is an immunotherapy agent. In some embodiments, the therapeutic agent is a radioimmunotherapy agent. In some embodiments, the therapeutic agent is a chemotherapeutic agent. In some embodiments, the therapeutic agent is an inhibitor. In some embodiments, the inhibitor is a small molecule. In some embodiments, the humanized anti-N-cadherin antibody is directed toward human N-cadherin.

### Humanized Anti-N-Cadherin Antibodies

In some embodiments, the invention includes compositions comprising an antibody that specifically binds to N-cadherin. In one embodiment, the anti-N-cadherin antibody is a polyclonal antibody. In another embodiment, the anti-N-cadherin antibody is a monoclonal antibody. In some embodiments, the anti-N-cadherin antibody is a chimeric antibody. In further embodiments, the anti-N-cadherin antibody is a humanized antibody. In some embodiments, the antibody is an antibody fragment. In preferred embodiments, the N-cadherin is human N-cadherin.

In some embodiments, binding of the antibody or the fragment of the antibody to human-N-cadherin is associated with a reduction in the activity of N-cadherin in the EMT signaling cascade in an intact organism. In some embodiments, the invention is a protein or a polypeptide capable of binding to human N-cadherin. In some embodiments, the antibody or antibody fragment; the protein or the polypeptide binds to a relevant portion or fraction or epitope of the human-N-cadherin; and the binding of the antibody, or the antibody fragment thereof, or the protein or the polypeptide to the relevant portion of the human-N-cadherin is associated with a reduction in the generation of N-cadherin in an intact organism.

In some embodiments, binding of the antibody or the fragment of the antibody to human-N-cadherin is associated with a reduction in the activity of N-cadherin in the EMT signaling cascade in an intact organism. In some embodiments, the invention is a protein or a polypeptide capable of binding to human N-cadherin. In some embodiments, the antibody or antibody fragment; the protein or the polypeptide binds to a relevant portion or fraction or epitope of the human-N-cadherin; and the binding of the antibody, or the antibody fragment thereof, or the protein or the polypeptide to the relevant portion of the human-N-cadherin is associated with a reduction in the activity of N-cadherin in an intact organism. In some embodiments, the relevant portion or fraction or epitope of the human N-cadherin is the extracellular domain 2. In some embodiments, the relevant portion or fraction or epitope of the human N-cadherin is the extracellular domain ¾.

In some embodiments, the peptide that binds to the relevant portion of the human-N-cadherin, is a cyclized peptide. In some embodiments, the peptide that binds to the relevant portion of the human-N-cadherin is a modified peptide. In some cases, the human-N-cadherin binding antibody or a N-cadherin binding antibody fragment thereof, is further conjugated to a protein, a peptide or another compound.

In one embodiment, the anti-N-cadherin antibody or an antigen-binding fragment thereof comprises at least one of the CDRs selected from the group consisting of: VH-CDR1: SEQ ID NO:5; VH-CDR2: SEQ ID NO:6; VH-CDR3: SEQ ID NO:7; VL-CDR1: SEQ ID NO:12; VL-CDR2: SEQ ID NO:13; and VL-CDR3: SEQ ID NO:14. In another embodiment, the anti-N-cadherin antibody comprises all of the CDRs of the group consisting of: VH-CDR1: SEQ ID NO:5; VH-CDR2: SEQ ID NO:6; VH-CDR3: SEQ ID NO:7; VL-CDR1: SEQ ID NO:12; VL-CDR2: SEQ ID NO:13; and VL-CDR3: SEQ ID NO:14.

In some embodiments, the humanized anti-N-cadherin antibody or an antigen binding fragment thereof comprises a heavy chain comprising the amino acid sequence of SEQ ID NO: 1. In some embodiments, the humanized anti-N-cadherin antibody comprises a light chain comprising the amino acid sequence of SEQ ID NO: 8. In another embodiment, the humanized anti-N-cadherin antibody is an antibody designated h1H7. The humanized anti-N-cadherin antibody designated h1H7 comprises a heavy chain comprising the amino acid sequence of SEQ ID NO:1 and a light chain comprising the amino acid sequence of SEQ ID NO: 8. In some embodiments, the humanized anti-N-cadherin antibody designated h1H7 is a chimeric antibody.

In one embodiment, the humanized anti-N-cadherin antibody or an antigen binding fragment thereof comprises at least one of the CDRs selected from the group consisting of: VH-CDR1: SEQ ID NO:18; VH-CDR2: SEQ ID NO:19; VH-CDR3: SEQ ID NO:20. In another embodiment, the humanized anti-N-cadherin antibody comprises all of the CDRs of the group consisting of: VH-CDR1: SEQ ID NO:18; VH-CDR2: SEQ ID NO:19; VH-CDR3: SEQ ID NO:20.

In some embodiments, the humanized anti-N-cadherin antibody comprises a heavy chain comprising the amino acid sequence of SEQ ID NO:15. In other embodiments, the humanized anti-N-cadherin antibody comprises a light chain comprising the amino acid sequence of SEQ ID NO:21. In another embodiment, the humanized anti-N-cadherin antibody is an antibody designated h2A9. The humanized anti-N-cadherin antibody designated h2A9 comprises a heavy chain comprising the amino acid sequence of SEQ ID NO:15 and a light chain comprising the amino acid sequence of SEQ ID NO:21.

In some embodiments, the anti-N-cadherin antibody comprises an antibody having about at least 80% amino acid identity with the CDR sequence described herein, listed in SEQ ID NOs 5-7, 12-14, 18-20 and 24-26.

In one embodiment, the current disclosure encompasses an anti-N-cadherin antibody having CDR sequences of about at least 80%, identity to the CDR sequences described above. The current disclosure encompasses an anti-N-cadherin antibody, or antigen binding fragment thereof, having CDR sequences of 81%, 82%, 83% 84%, 85%, 86%, 87%, 88%, 89%, 90%, 91%, 92%, 93%, 94%, 95%, 96%, 97% and 99% amino acid sequence identity with the CDR sequences described herein. In one embodiment, the antibody against human N-cadherin has a heavy chain variable (vH) region and a light chain variable (vL) region, wherein the vH region has an amino acid sequence that is more than 90% identical to SEQ ID NO 1 and wherein the vL region has an amino acid sequence that is more than 90% identical to SEQ ID NO 8. In some embodiments the antibody or the antibody fragment is modified. In some embodiments the modifications include fusion of the antibody or the antigen-binding fragment thereof with portions of another protein, or a protein fragment. In some embodiments the antibody or the antibody fragment thereof of the invention is modified to increase the circulating half-life of the same in vivo. For example, the antibody of the fragment may be fused with an FcRn molecule, which is also known as neonatal Fc receptor to stabilize the antibody in vivo. (Nature Reviews Immunology 7:715-725). One of skill in the art would be able to prepare human-N-cadherin binding single chain variable fragment (scFv), comprising at least one specific CDR sequence selected from SEQ ID NOs 12-14, 18-20 and 24-26. An scFv may comprise heavy chain variable region sequences designated in SEQ ID NOs 5-7 and 18-20, and light chain variable regions designated in SEQ ID NOs 12-14 and 24-26. CDR sequences incorporated within the scFv having amino acid sequence identity of 80%, 81%, 82%, 83%, 84%, 85% 86%, 87%, 88%, 89%, 90%, 91%, 92%, 93%, 94%, 95%, 96%, 97%, 98%, and 99% to the CDR sequences described in the present disclosure are encompassed within the scope of the present disclosure.

### Therapeutic Agent Combination

In one aspect, the present invention relates to a composition comprising a therapeutic agent in combination with a humanized anti-human N-cadherin antibody of the invention. In one embodiment, the therapeutic agent comprises an “effector” moiety. In one embodiment, the therapeutic agent comprises an inhibitor.

In one embodiment, the anti-human N-cadherin antibody of the invention is conjugated to an “effector” moiety. The effector moiety can be any number of molecules, including labeling moieties such as radioactive labels or fluorescent labels, or can be a therapeutic moiety. In one aspect, the antibody modulates the activity of the protein. Such effector moieties include, but are not limited to, an anti-tumor drug, a toxin, a radioactive agent, a cytokine, a second antibody or an enzyme. Further, the invention provides an embodiment wherein the antibody of the invention is linked to an enzyme that converts a prodrug into a cytotoxic agent. The immunoconjugate can be used for targeting the effector moiety to a N-cadherin positive cell, particularly cells, which express the N-cadherin protein. Such differences can be readily apparent when viewing the bands of gels with approximately similarly loaded with test and controls samples. Examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

In one embodiment, inhibitors are used in combination with a humanized anti-N-cadherin antibody of the invention to prevent expression of or activity of N-cadherin. In some embodiments, the inhibitors are used to prevent binding of proteins to N-cadherin. In some embodiments, the inhibitors of N-cadherin expression are regulators of transcription and translation of N-cadherin, including but are not limited to siRNA, antisense nucleic acids, ribozymes, small molecules, and antagonists. In some embodiments, the regulators of transcription factor activity are enzymes including but not limited to kinases. In some embodiments, regulators of transcription include by are not limited to polymerases, acetyltransferases, histone deacetylases, and methylases.

siRNA

In one embodiment, the therapeutic agent comprising an “effector” moiety is an siRNA. In one embodiment, the siRNA is used to decrease the activity of N-cadherin. In one embodiment, the siRNA is used to decrease the level of N-cadherin expression or activation. In one embodiment, the siRNA is used to decrease the level of one or more epicenter regulator protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, P A (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of the desired transcription factor at the protein level using RNAi technology. In doing so, the present invention includes methods of decreasing the activity of one or more epicenters.

In other related aspects, the invention includes an isolated nucleic acid encoding an inhibitor, wherein an inhibitor such as an siRNA or antisense molecule, inhibits the desired N-cadherin activity, one or more molecules, one or more proteins binding thereto, a derivative thereof, a regulator thereof, or a downstream effector, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein. In another aspect of the invention, the desired N-cadherin activity, one or more molecules, one or more transcription factors binding thereto, or a regulator thereof, can be inhibited by way of inactivating and/or sequestering the one or more N-cadherin molecules or activity thereof. As such, inhibiting the effects of the epicenter or one or more transcription factors binding thereto can be accomplished by using a transdominant negative mutant.

In another aspect, the invention includes a vector comprising an siRNA or antisense polynucleotide. Preferably, the siRNA or antisense polynucleotide is capable of inhibiting the expression of the one or more N-cadherin molecules, activity thereof, or other proteins involved in the regulation of the one or more N-cadherin molecules or activity. The incorporation of a desired polynucleotide into a vector and the choice of vectors is well-known in the art as described in, for example, Sambrook et al., supra, and Ausubel et al., supra, and elsewhere herein.

The siRNA or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA or antisense polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Antisense Nucleic Acids

In one embodiment of the invention, an antisense nucleic acid sequence which is expressed by a plasmid vector is used to inhibit one or more N-cadherin molecules, or activity thereof. The antisense expressing vector is used to transfect a mammalian cell or the mammal itself, thereby causing reduced expression of one or more N-cadherin molecules, or activity thereof, endogenous expression of the one or more N-cadherin molecules.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243). Compositions and methods for the synthesis and expression of antisense nucleic acids are as described elsewhere herein.

Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit a desired one or more N-cadherin molecules, regulators thereof, or activity thereof. Ribozymes useful for inhibiting the expression of a target molecule may be designed by incorporating target sequences into the basic ribozyme structure which are complementary, for example, to the mRNA sequence of the N-cadherin antibody of the present invention. Ribozymes targeting a desired N-cadherin antibody may be synthesized using commercially available reagents (Applied Biosystems, Inc., Foster City, CA) or they may be genetically expressed from DNA encoding them.

Small Molecules

When the inhibitor in combination with the one or more humanized anti-human N-cadherin antibodies of the invention is an is a small molecule, a small molecule agonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In one embodiment, the small molecule is able to inhibit one or more N-cadherin molecules, regulators thereof, or activity thereof

Antagonist

In another aspect of the invention, the inhibitor in combination with a humanized anti-human N-cadherin antibody of the invention is an antagonist of N-cadherin such that one or more N-cadherin molecules, regulators thereof, or activity thereof, can be inhibited by way of inactivating and/or sequestering the one or more N-cadherin molecules, regulators thereof, or activity thereof. As such, inhibiting the effects of one or more N-cadherin molecules, regulators thereof, or activity thereof can be accomplished by using a transdominant negative mutant. Alternatively, regulators of one or more N-cadherin molecules, or activity thereof, otherwise known as an antagonist to the one or more N-cadherin molecules, regulators thereof, or activity thereof may be used. In one embodiment, the antagonist is a protein and/or compound having the desirable property of interacting with a binding partner of the one or more N-cadherin molecules, regulators thereof, or activity thereof, and thereby competing with the corresponding protein. In another embodiment, the antagonist is a protein and/or compound having the desirable property of interacting with the one or more N-cadherin molecules, regulators thereof, or activity thereof and thereby sequestering the one or more N-cadherin molecules, regulators thereof, or activity thereof.

### Methods

In one embodiment, the invention is a method of treating an N-cadherin-mediated disease or disorder in an individual, comprising the step of administering to said individual a humanized anti-N-cadherin antibody, thereby selectively inhibiting the effects of N-cadherin protein. Examples of N-cadherin-mediated pathologies that can be treated using the compositions and methods of the invention include, but are not limited to cancer, prostate cancer, hormone-refractory disease, hormone-refractory prostate cancer, bladder cancer, carcinoma, melanoma, breast cancer, adrenal tumors, or any combinations thereof.

In some embodiments, the composition treats or prevents an N-cadherin-mediated disease or disorder by inhibiting N-cadherin-mediated signaling. In some embodiments, N-cadherin mediated signaling is epithelial to mesenchymal transition.

In some embodiments, the invention relates to methods for diagnosing cancer in a subject wherein the cancer includes but is not limited to prostate cancer, bladder cancer, carcinoma, melanoma, breast cancer, adrenal tumors, and combinations thereof.

In some embodiments, the invention provides a method of treating cancer, particularly a cancer which expresses N-Cadherin, or of inhibiting the growth of a cancer cell expressing a N-Cadherin protein by treating a subject or contacting the cancer cell with an antibody or fragment thereof that recognizes and binds the N-Cadherin protein in an amount effective to inhibit the growth of the cancer cell. In some embodiments, the cancer cell is a prostate cancer cell or a bladder cancer cell.

In any of the embodiments discussed herein, a chemotherapeutic drug and/or radiation therapy can be administered further. In some embodiments, the patient also receives hormone antagonist therapy. The contacting of the patient with the antibody or antibody fragment, can be by administering the antibody to the patient intravenously, intraperitoneally, intramuscularly, intratumorally, or intradennally. In some embodiments, the patient has a urogenital cancer (e.g., bladder cancer, prostate cancer). In some embodiments of the above, the patient suffers from prostate cancer and optionally further receives patient hormone ablation therapy. In some embodiments, the contacting comprises administering the antibody of the invention directly into the cancer or a metastasis of the cancer.

In some embodiments, the invention provides a method of treating a cancer patient. The method generally comprises (a) obtaining a test tissue sample from an individual at risk of having a cancer that expresses an N-cadherin protein; (b) determining the presence or absence or amount of the N-cadherin protein in the test tissue sample in comparison to a control tissue sample from an individual known to be negative for the cancer; thereby diagnosing the cancer that expresses an N-cadherin protein, wherein the N-cadherin protein is expressed at normal or low levels, or is expressed by a subset of cells and is not overexpressed; (c) determining whether a cancer is likely to become invasive, metastasize, hormone independent, or refractory treatment; (d) administering a chemotherapeutic agent, an immunotherapeutic agent, hormonal therapy, or radiotherapy according to whether there is an increased likelihood of the cancer becoming invasive, metastasizing, hormone independent, or refractory to treatment.

In some embodiments, the chemotherapeutic agent can be selected from the group consisting of ricin, ricin A-chain, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, maytansinoids, and glucocorticoidricin.

### Screening Assays

The present invention has application in various screening assays, including, determining whether a candidate humanized anti-N-cadherin antibody can inhibit N-cadherin activity.

In some embodiments, the level of N-cadherin expression or activity in the presence of the candidate humanized anti-N-cadherin antibody is compared with N-cadherin expression or activity detected in a positive comparator control. The positive comparator control comprises N-cadherin activation in the absence of added test compound. In some embodiments, the candidate humanized anti-N-cadherin antibody is identified as an inhibitor of N-cadherin when the N-cadherin activity in the presence of the candidate humanized anti-N-cadherin antibody is less than about 70% of the N-cadherin activity detected in a positive comparator control; this corresponds to greater than about 30% inhibition of N-cadherin activity in the presence of the test compound. In other embodiments, the candidate humanized anti-N-cadherin antibody is identified as an inhibitor of the N-cadherin when the N-cadherin activity in the presence of the candidate humanized anti-N-cadherin antibody is less than about 80% of the N-cadherin activity detected in a positive comparator control; this corresponds to greater than about 20% inhibition of N-cadherin activity in the presence of the test compound. In still other embodiments, the candidate humanized anti-N-cadherin antibody is identified as an inhibitor of the N-cadherin when the N-cadherin activity in the presence of the candidate humanized anti-N-cadherin antibody is less than about 90% of the N-cadherin activity detected in a positive N-cadherin control; this corresponds to greater than about 10% inhibition of N-cadherin activity in the presence of the test compound. In some embodiments, the level of N-cadherin inhibition by the candidate humanized anti-N-cadherin antibody is compared with the level of inhibition detected in a negative comparator control.

A variety of immunoassay formats, including competitive and non-competitive immunoassay formats, antigen capture assays, two-antibody sandwich assays, and three-antibody sandwich assays are useful methods of the invention (Self et al., 1996, Curr. Opin. Biotechnol. 7:60-65). The invention should not be construed to be limited to any one type of known or heretofor unknown assay, provided that the assay is able to detect the inhibition of N-cadherin.

Enzyme-linked immunosorbent assays (ELISAs) are useful in the methods of the invention. An enzyme such as, but not limited to, horseradish peroxidase (HRP), alkaline phosphatase, beta-galactosidase or urease can be linked, for example, to an anti-N-cadherin antibody or to a secondary antibody for use in a method of the invention. A horseradish-peroxidase detection system may be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which may be used with the chromogenic substrate p-nitrophenyl phosphate to yield a soluble product readily detectable at 405 nm. Similarly, a beta-galactosidase detection system may be used with the chromogenic substrate o-nitrophenyl-beta-D-galactopyranoside (ONPG) to yield a soluble product detectable at 410 nm. Alternatively, a urease detection system may be used with a substrate such as urea-bromocresol purple (Sigma Immunochemicals, St. Louis, MO). Useful enzyme-linked primary and secondary antibodies can be obtained from any number of commercial sources.

Chemiluminescent detection is also useful for detecting the inhibition of the AP. Chemiluminescent secondary antibodies may be obtained from any number of commercial sources.

Fluorescent detection is also useful for detecting the inhibition of the AP. Useful fluorochromes include, but are not limited to, DAPI, fluorescein, Hoechst 33258, R-phycocyanin, B-phycoerythrin, R-phycoerythrin, rhodamine, Texas red and lissamine-Fluorescein- or rhodamine-labeled antibodies.

Radioimmunoassays (RIAs) are also useful in the methods of the invention. Such assays are well known in the art, and are described for example in Brophy et al. (1990, Biochem. Biophys. Res. Comm. 167:898-903) and Guechot et al. (1996, Clin. Chem. 42:558-563). Radioimmunoassays are performed, for example, using Iodine-125-labeled primary or secondary antibody (Harlow et al., supra, 1999).

A signal emitted from a detectable antibody is analyzed, for example, using a spectrophotometer to detect color from a chromogenic substrate; a radiation counter to detect radiation, such as a gamma counter for detection of Iodine-125; or a fluorometer to detect fluorescence in the presence of light of a certain wavelength. Where an enzyme-linked assay is used, quantitative analysis is performed using a spectrophotometer. It is understood that the assays of the invention can be performed manually or, if desired, can be automated and that the signal emitted from multiple samples can be detected simultaneously in many systems available commercially.

The methods of the invention also encompass the use of capillary electrophoresis based immunoassays (CEIA), which can be automated, if desired. Immunoassays also may be used in conjunction with laser-induced fluorescence as described, for example, in Schmalzing et al. (1997, Electrophoresis 18:2184-2193) and Bao (1997, J. Chromatogr. B. Biomed. Sci. 699:463-480). Liposome immunoassays, such as flow-injection liposome immunoassays and liposome immunosensors, may also be used according to the methods of the invention (Rongen et al., 1997, J. Immunol. Methods 204:105-133).

Quantitative western blotting may also be used to determine the level of N-cadherin inhibition in the methods of the invention. Western blots are quantified using well known methods such as scanning densitometry (Parra et al., 1998, J. Vasc. Surg. 28:669-675).

### Methods of Administration

The methods of the invention comprise administering a therapeutically effective amount of at least one humanized anti-N-cadherin antibody, or binding fragment thereof, to an individual identified as having an N-cadherin-mediated disease or disorder. In one embodiment the individual is a mammal. In one embodiment the individual is a human. In various embodiments, the at least one anti-N-cadherin antibody, or binding fragment thereof, is administered locally, regionally, or systemically.

The methods of the invention can comprise the administration of at least one humanized anti-N-cadherin antibody, or binding fragment thereof, but the present invention should in no way be construed to be limited to the anti-N-cadherin antibodies described herein, but rather should be construed to encompass any anti-N-cadherin antibody, both known and unknown, that diminish and reduce N-cadherin activation.

The method of the invention comprises administering a therapeutically effective amount of at least one anti-N-cadherin antibody, or binding fragment thereof, to an individual wherein a composition of the present invention comprising at least one anti-N-cadherin antibody, or binding fragment thereof, either alone or in combination with at least one other therapeutic agent. The invention can be used in combination with other treatment modalities, such as, for example anti-inflammatory therapies, and the like. Examples of anti-inflammatory therapies that can be used in combination with the methods of the invention include, for example, therapies that employ steroidal drugs, as well as therapies that employ non-steroidal drugs.

The method of the invention comprises administering a therapeutically effective amount of a humanized anti-N-cadherin antibody, or an antigen-binding fragment thereof, to a subject. In some embodiments, the invention encompasses a method of treatment of N-cadherin related diseases involving dysregulation of the N-cadherin signaling by administering a therapeutically effective amount of an antibody of the invention, or a therapeutically effective amount of an antibody fragment thereof, such that a reduction of N-cadherin activity is effected in the subject. In some embodiments, the invention encompasses a method of treatment of N-cadherin related diseases involving dysregulation of N-cadherin signaling by administering a therapeutically effective amount of an antibody or an antibody fragment. In some embodiments, the invention encompasses a method of treatment of N-cadherin related diseases involving dysregulation of N-cadherin signaling by administering to a subject an effective amount of an antibody, an antibody fragment, a polypeptide, a peptide, a conjugated peptide or a cyclized peptide, such that the N-cadherin activation pathway activation is reduced in the subject. In some embodiments, the method of treatment encompasses administering to a subject a systemically effective dose of an antibody or an antibody fragment, whereby systemic reduction of N-cadherin activity is effected in the subject.

Administration of a humanized anti-N-cadherin antibody, or binding fragment thereof, in a method of treatment of the invention can be achieved in a number of different ways, using methods known in the art. The therapeutic and prophylactic methods of the invention thus encompass the use of pharmaceutical compositions comprising an anti-N-cadherin antibody. The pharmaceutical compositions useful for practicing the invention may be administered to deliver a dose of between about 1 ng/kg/day, about 5 ng/kg/day, about 10 ng/kg/day, about 25 ng/kg/day, about 50 ng/kg/day, about 100 ng/kg/day, about 500 ng/kg/day, about 1 μg/kg/day, about 5 μg/kg/day, about 10 μg/kg/day, about 25 μg/kg/day, about 50 μg/kg/day, about 100 μg/kg/day, about 500 μg/kg/day, about 1 mg/kg/day, about 5 mg/kg/day, about 10 mg/kg/day, about 25 mg/kg/day, about 50 mg/kg/day and 100 mg/kg/day. In one embodiment, the invention administers a dose which results in a concentration of the anti-N-cadherin antibody of the present invention of about 1 pM, about pM, about 100 pM, about 1 nM, about 10 nM, about 100 nM, about 1 μM, about 2 μM, about 3 μM, about 4 μM, about 5 μM, about 6 μM, about 7 μM, about 8 μM, about 9 μM and about 10 μM in an individual. In another embodiment, the invention envisions administration of a dose which results in a concentration of the anti-N-cadherin antibody of the present invention between about 1 pM, about 10 pM, about 100 pM, about 1 nM, about 10 nM, about 100 nM, about 1 μM, about 2 μM, about 3 μM, about 4 μM, about 5 μM, about 6 μM, about 7 μM, about 8 μM, about 9 μM and about 10 μM in the plasma of an individual.

Typically, dosages which may be administered in a method of the invention to a subject, in some embodiments a human, range in amount from 0.5 μg to about 50 mg per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 μg to about 1 mg per kilogram of body weight of the subject.

The antibody of the invention may be administered to a subject as frequently as several times daily, or it may be administered less frequently, such as once a day, twice a day, thrice a day, once a week, twice a week, thrice a week, once every two weeks, twice every two weeks, thrice every two weeks, once a month, twice a month, thrice a month, or even less frequently, such as once every several months or even once or a few times a year or less. The frequency of the dose will be readily apparent to the skilled artisan and will depend upon any number of factors, such as, but not limited to, the type and severity of the disease being treated, the type and age of the subject, etc. The formulations of the pharmaceutical compositions may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to subjects of all sorts. Modification of pharmaceutical compositions suitable for administration to humans in order to render the compositions suitable for administration to various subjects is well understood, and the ordinarily skilled veterinary pharmacologist can design and perform such modification with merely ordinary, if any, experimentation. Individuals to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intraocular, intramuscular, intradermal and intravenous routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. A unit dose is discrete amount of the pharmaceutical composition comprising a predetermined amount of the active ingredient. The amount of the active ingredient is generally equal to the dosage of the active ingredient which would be administered to an individual or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the individual treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and in some embodiments from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. In some embodiments, the droplets provided by this route of administration have an average diameter in the range from about 0.1 to about 200 nanometers.

The formulations are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more additional ingredients.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more additional ingredients. Alternately, formulations suitable for buccal administration may comprise a powder or an aerosolized or to atomized solution or suspension comprising the active ingredient. In some embodiments, such powdered, aerosolized, or aerosolized formulations, when dispersed, have an average particle or droplet size in the range from about 0.1 to about 200 nanometers, and may further comprise one or more additional ingredients.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Kits

The invention also includes a kit comprising a humanized anti-N-cadherin antibody, or combinations thereof, of the invention and an instructional material which describes, for instance, administering the anti-N-cadherin antibody, or combinations thereof, to an individual as a therapeutic treatment or a non-treatment use as described elsewhere herein. In an embodiment, this kit further comprises a (preferably sterile) pharmaceutically acceptable carrier suitable for dissolving or suspending the therapeutic composition, comprising an anti-N-cadherin antibody, or combinations thereof, of the invention, for instance, prior to administering the antibody to an individual. Optionally, the kit comprises an applicator for administering the antibody.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore and are not to be construed as limiting in any way the remainder of the disclosure.

### Example 1. Humanized Antibodies Recognizing the Extracellular Domain of N-Cadherin

N-cadherin consists of five extracellular cadherin repeats, a transmembrane region and a highly conserved cytoplasmic tail. While the cytoplasmic tail mediates binding to catenins, which in turn interact with the actin cytoskeleton, the extracellular domains mediate homophilic interactions between adjacent cells.

Murine monoclonal antibodies targeting N-cadherin domains 2 (1H7), and ¾ (2A9), respectively, have shown inhibitory effects on multiple prostate cancer models in vivo (Tanaka et al. 2010).

Humanized antibodies that recognize human N-cadherin, described herein, provide improved agents and methods for diagnosis and treatment of diseases associated with expression of N-cadherin. The identification and characterization of these antibodies has led to the design of humanized antibodies for therapeutic use. In particular the variable regions of the antibodies were humanized, providing humanized immunoglobulin domains for intact humanized antibodies or antibody fragments of the featured antibodies.

The methods used herein are described below.

### Humanization of Murine Antibodies Targeting N-Cadherin

Humanized versions of the N-cadherin murine antibodies 1H7 and 2A9 were generated by grafting all 6 complementarity determining regions (CDRs) onto human variable germline genes (see Table 1).

The amino acid sequences were back-translated into nucleotide sequences. Codon-optimized DNA (codon usage and G/C content adaption for Homo sapiens) encoding the humanized variable domains connected by a 15 glycine-rich amino acid-linker (VH-(G4S)3-VL, scFv) was synthesized by GeneArt (Life Technologies™) and were supplied in plasmid pMA-T containing appropriate cloning sites. Plasmids were digested using restriction enzymes AgeI and NotI and the resulting fragment was inserted into vector pSECTag2A (AgeI) downstream of the Igκ-leader sequence and adding a C-terminal His-tag.

### Cloning of Humanized Full Length IgG

To generate full length monoclonal antibodies based on the humanized variable domains, cloning plasmids were used that contain the constant regions of the human kappa light chain and human Ig gamma 1 heavy chain (pFUSE2ss-CLIg-hK and pFUSEss-CHIg-hG1, InvivoGen). The light and heavy chain variable domains were cloned upstream of the respective constant region. Small scale test productions were conducted using double transfected 293-F cells (Gibco™ Invitrogen, Cat. No. 11625-019). Large scale transient expression in suspension 293-6E cultures and Protein A purification was outsourced to GenScript.

### Cloning of Chimeric N-Cadherin-Fc Fusion Proteins

DNA encoding the extracellular domains of human N-cadherin (P19022 UniProtKB, Cadherin-2, CDH2 Human) ECD1-3 (aa160-497) fused to the murine gamma 2a Fc region (Fc mγ2a) and ECD4-5 (aa498-714) were synthesized by GeneArt. The fragment encoding ECD1-3-Fc was cloned into pSECTag2A (AgeI) via restriction sites AgeI and XhoI. The resulting plasmid pSECTag2A ECD1-3-Fc was digested using enzymes AgeI and NotI to replace the fragment encoding ECD1-3 with the respective single domains (ECDs 1-5) or successive domains (ECD34, ECD45).

### Protein Production

293-F cells were transfected with plasmid DNA using Lipofectamine™ 2000 (Invitrogen) according to the manufacturer's instructions. Stable cell pools were selected in the presence of Zeocin™ Selection Reagent (life technologies) or in case of the co-transfected pFUSEss plasmids in the presence of both zeocin and blasticidin.

For both stable and transient protein expression the growth medium was replaced with reduced serum medium (Opti-MEM®, ThermoFisher Scientific) and supernatant was collected every 3-4 days.

### Protein Purification

Recombinant histidine-tagged scFv proteins were purified by immobilized metal ion affinity chromatography using HisTrap HP columns in an Äkta chromatography system (GE Healthcare Life Sciences). The columns prepackaged with Ni Sepharose were equilibrated with PBS, 20 mM imidazole and the concentrated cell culture supernatant was loaded. Unbound proteins were washed off using at least 10 column volumes PBS, 20 mM imidazole before bound protein was eluted by increasing the imidazole concentration (gradient 20-500 mM imidazole). Eluted protein containing fractions were dialyzed twice against PBS.

Intact IgG from small scale test production and recombinant human N-cadherin extracellular domain Fc fusion proteins were purified by affinity chromatography using HiTrap Protein A HP columns in the Äkta chromatography system. Columns were equilibrated using 5 column volumes of binding buffer (20 mM sodium phosphate pH 7.0). Prior to loading onto the column supernatants were concentrated and adjusted to pH 8.0-9.0 by adding 1/10 volume of 1M Tris-HCl pH 9.0. Unbound protein was removed by washing the column with binding buffer. Bound protein was eluted with 100 mM citric acid pH 3.0, neutralized by adding 1/10 volume 1M TrisHCl pH 9.0 and protein containing fractions were dialyzed against PBS.

### Biochemical Characterization

Purified proteins were analyzed by SDS-PAGE and Western blot for purity and integrity. Western Blot was also used to test antigen specificity and epitope mapping. Therefor commercial and in-house produced N-cadherin protein were blotted and recombinant antibody fragments tested for binding to the various extracellular domains of human N-cadherin.

Antigen binding was further test by performing saturation binding on immobilized antigen in ELISA.

### In Vivo Therapeutic Activity

Human prostate cancer cell line derivatives of the LNCaP cell line were injected subcutaneously into male castrated SCID mice and allowed to grow to a volume of 100 mm3. Treatment with anti-N-cadherin antibodies (parental mouse or humanized versions) was repeated 3 times a week at a 10 mg/kg dose administered intraperitoneally. Control groups received isotype antibodies and PBS treatment. Tumor volumes were caliper measured and experiments were terminated when tumors reached more than 1500 mm3.

The results are now described.

### Humanization

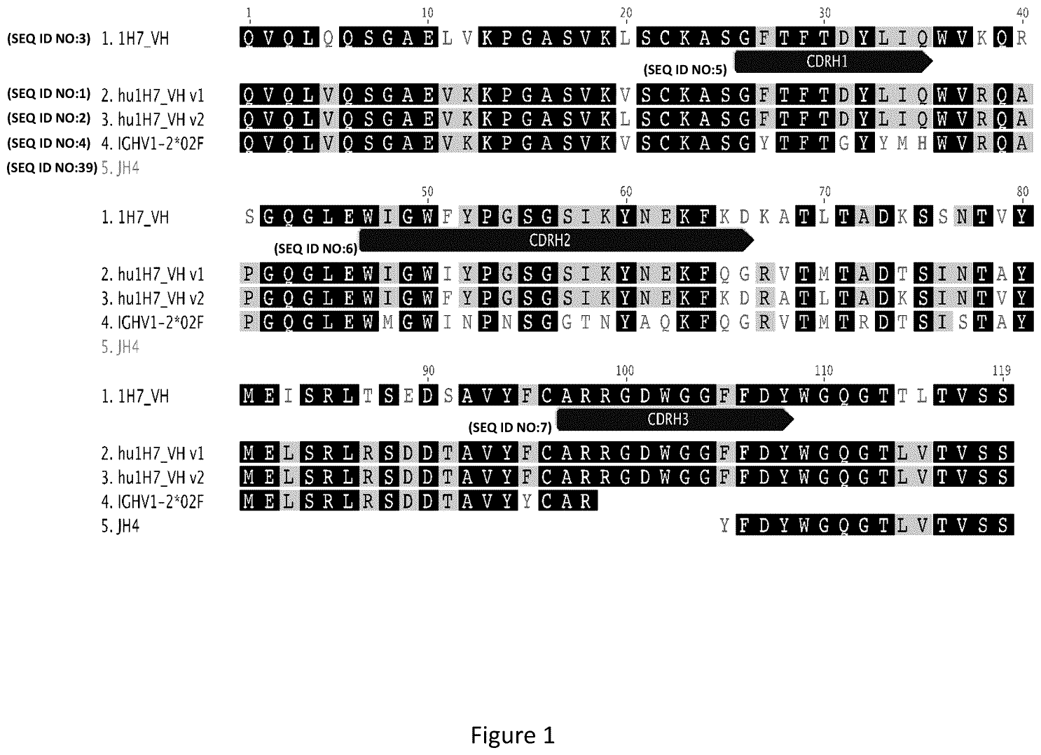

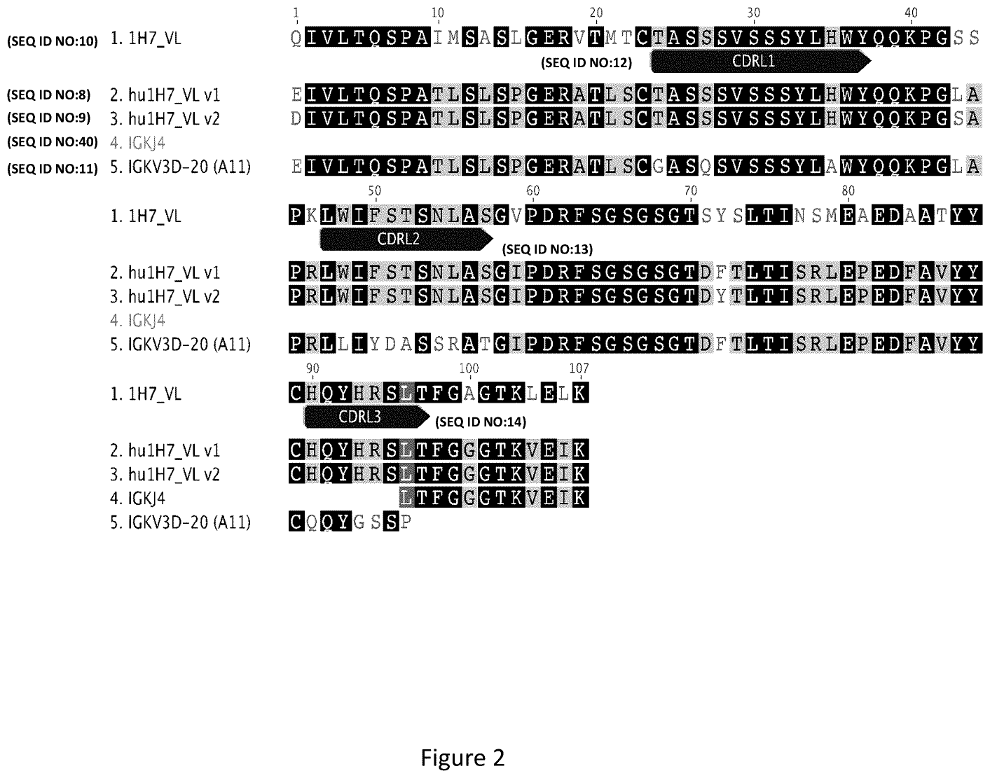

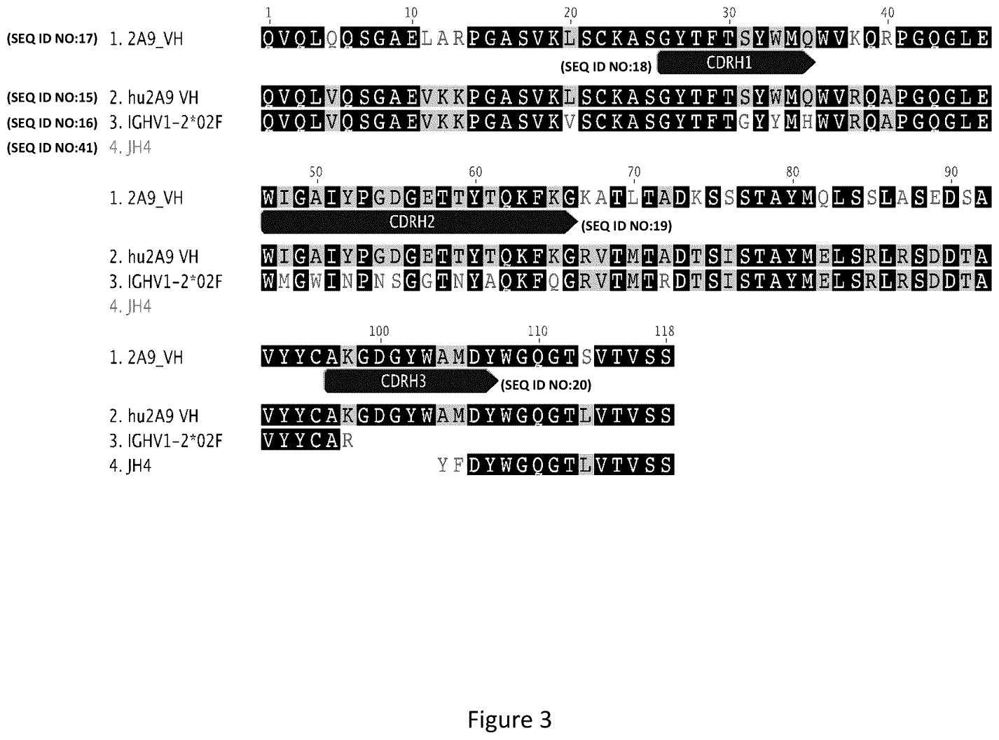

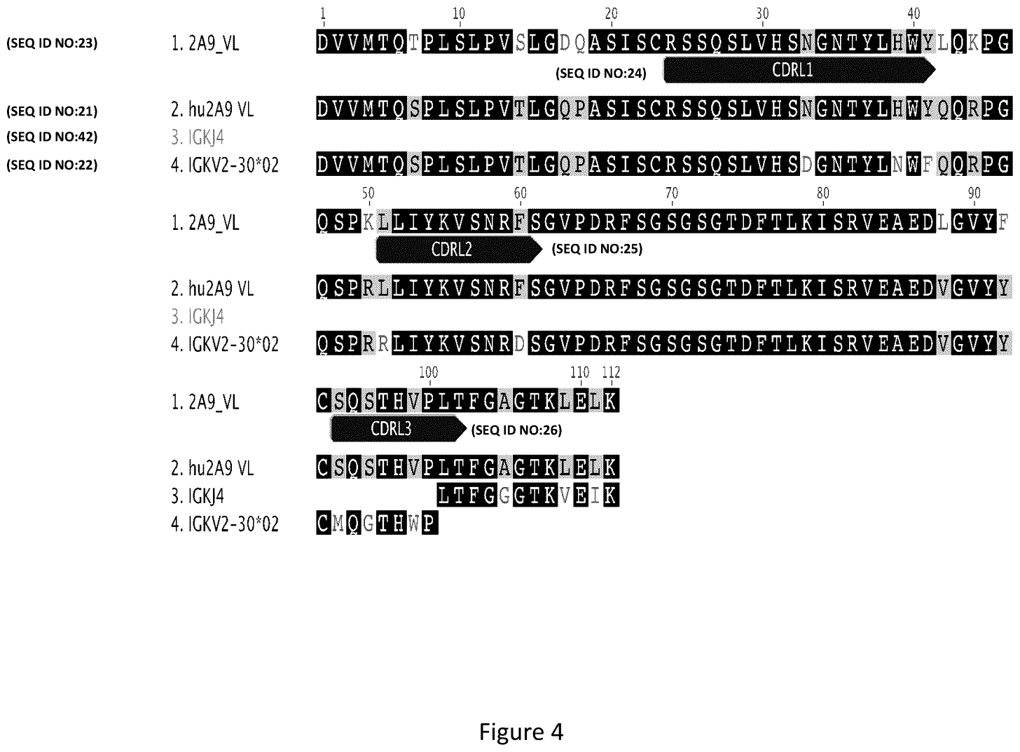

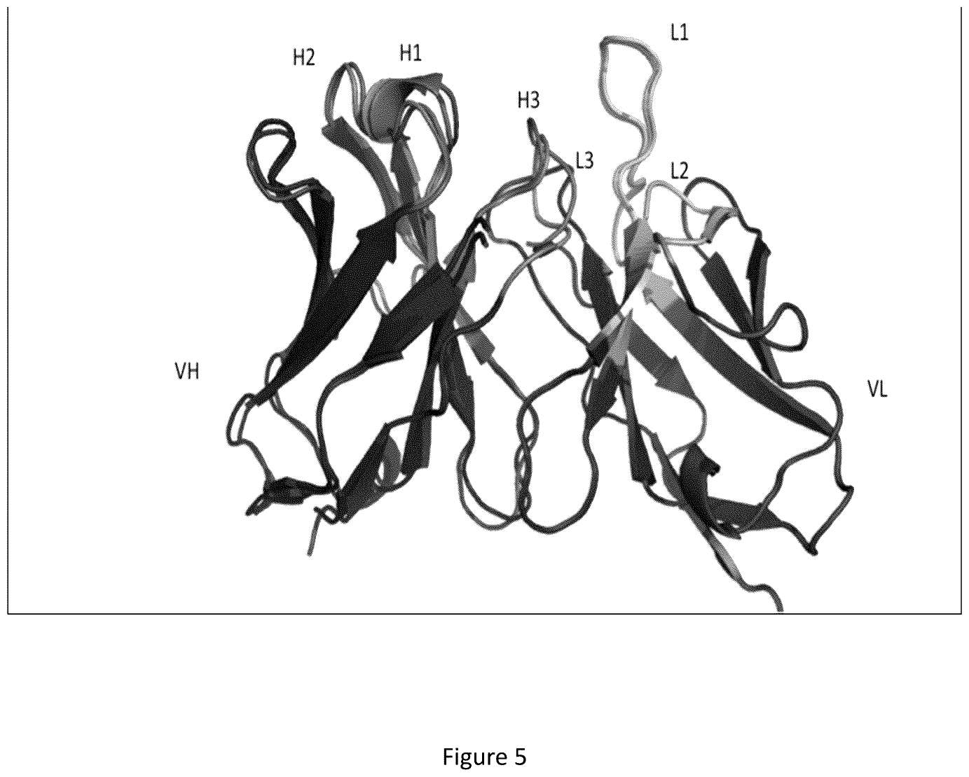

The design of the humanized antibody is the critical step in order to retain antigen specificity and binding affinity. The integrated database and analysis suite abYsis (Version 2.7.4) was used to number (Chothia numbering) and analyze the variable domains derived from hybridomas 1H7 and 2A9. Framework regions (FR) and complementarity determining regions (CDR) were defined based on the contact definition (residues which take part in interaction with antigens, based on the analysis of available complex crystal structures). Residues that are located at the VH/VL interface and in the Vernier zone underlying the CDRs were identified based on literature (Foote and Winter 1992). Canonical class alignment results and humanness scores are shown in Table 1. The abYsis suite was also used for alignment of the query sequence (mouse variable) to the most similar V, D, and J human germline gene segments (Data Source: NCBI Germline and V-BASE) in order to select the human light and heavy chain regions to serve as template for CDR-grafting. The most appropriate human sequences (most similar sequence, canonical class and highest Z-score, humanness) were chosen for virtual CDR-grafting (FIG. 1 through FIG. 4) and superimposed modeling using Web Antibody Modeling (WAM, http://antibody.bath.ac.uk/) (FIG. 5).

TABLE 1
Analysis of the variable regions of N-cadherin targeting antibodies.

| | VH 1H7 | VL 1H7 | VH 2A9 | VL 2A9 |
|---|---|---|---|---|
| Canonical class | | | | |
| alignment | | | | |
| CDR1 | 1/10A | ?/12A | 1/10A | 4/16A |
| | | (mismatch L2, | | (mismatch L2) |
| | | L93) | | |
| CDR2 | 2/10A | 1/7A | 2/10A | 1/7A |
| CDR3 | | ?/8B | | 1/9A |
| | | (mismatch L89) | | |
| Homologous | IGHV1-2*02F/ | IGKV3D-20 | IGHV1-2*02/ | IGKV2-30*02/ |
| human germline | IGHJ4*01 | (A11)/IGKJ4*02 | IGHJ4*01 | IGKJ4*01 |
| Humanness | | | | |
| (Z-score) | | | | |
| Murine | −1.6 | −0.9 | −1.1 | −1.3 |
| Humanized (v 1) | −0.4 | 0.2 | −0.1 | −1.0 |
| Humanized (v 2) | −0.7 | 0.3 | | |
| Retained mouse | 22 (v1)/30 (v2) | 13 (v1)/16 (v2) | 23 | 9 |
| residues | | | | |

The calculated Z-scores (Abhinandan and Martin 2007) represent a similarity to known human sequences with a score of zero representing average humanness and positive scores being more representative of human sequences. The calculated Z-scores for humanness were increased for all sequences after humanization.

ScFv hu2A9 and hu1H7

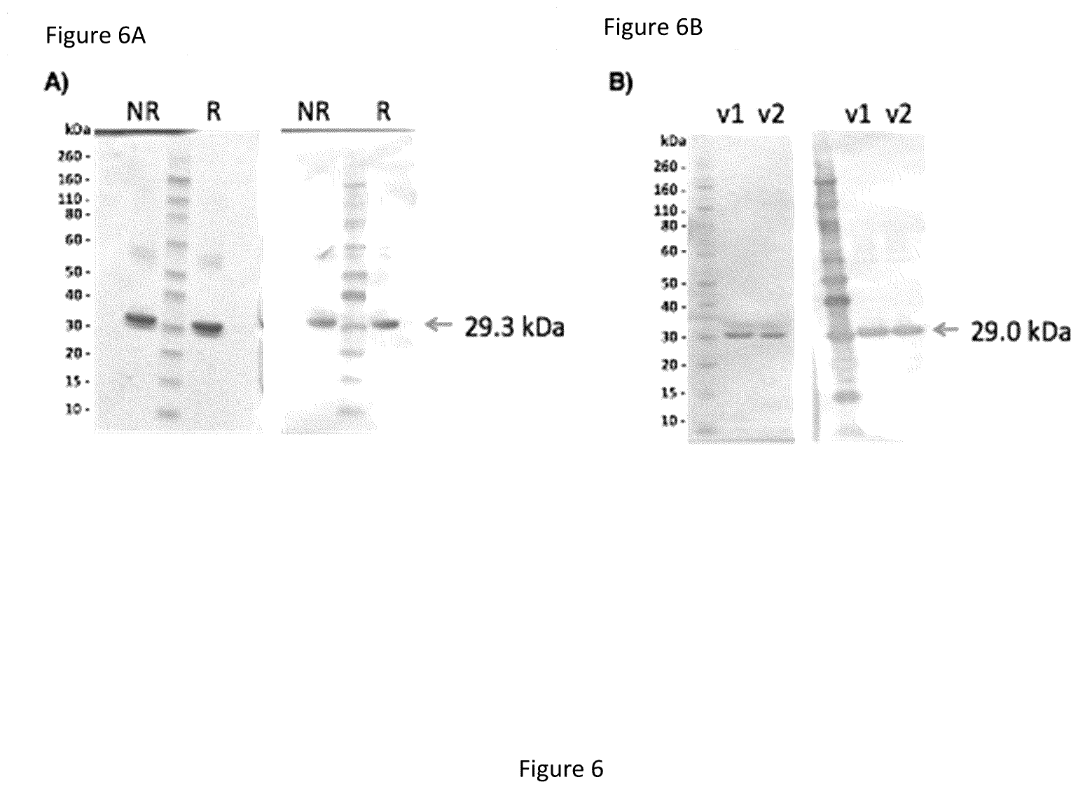

Codon optimized VH and VL domains were expressed as scFv fragments and purified from mammalian cell culture supernatant. SDS-PAGE analysis showed a single band with an apparent molecular mass of approximately 30 kDa (calculated MW 29.3 kDa for hu2A9 scFv and 29.0 kDa for hu1H7 scFv). Immunoblot analysis using the HisDetector Western Blot Kit (KPL Cat. No. 25-00-01) confirmed the identity of the scFvs (FIG. 6).

### Antigen Specificity and Epitope Mapping

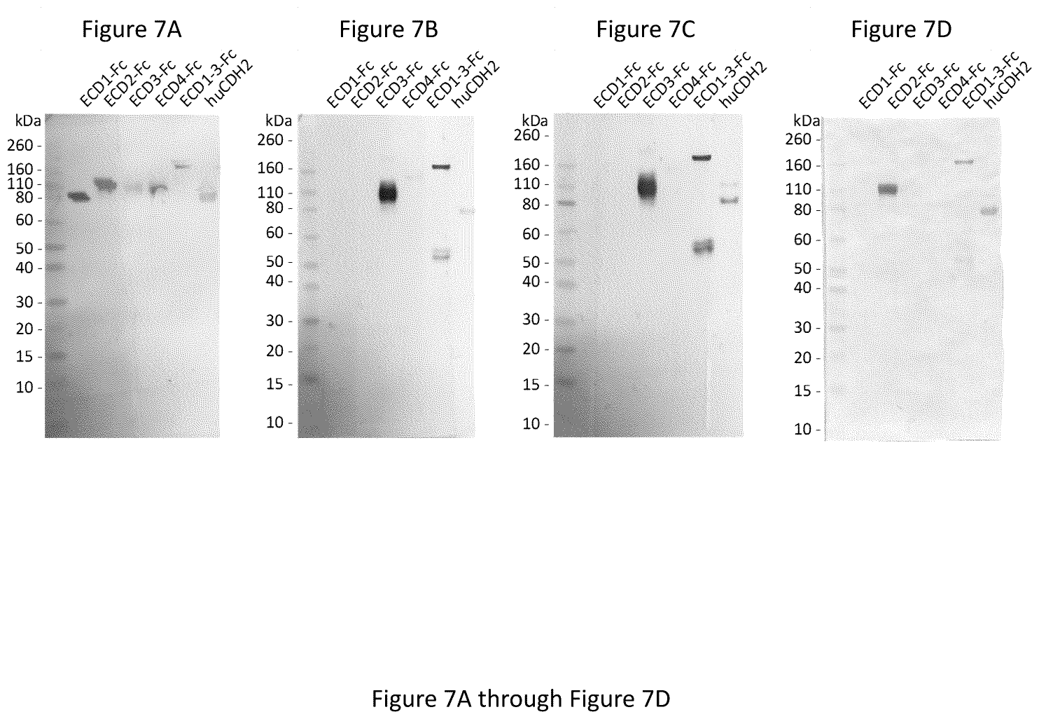

The purified scFv fragments were used to detect recombinant human N-cadherin ECD-Fc fusion proteins and recombinant human CDH2/NCAD (Sino Biological Inc., Cat. No. 11039-H08H, extracellular domain of human CDH2 (Met1-Ala724)) by immunoblot (FIG. 7).

The humanized 2A9 scFv bound to ECD3-Fc, ECD1-3-Fc and full length CDH2 similar to mo2A9 scFv composed of the parental mouse variable domains confirming successful humanization and retained antigen specificity to domain 3 of N-cadherin. The epitope of humanized 1H7 scFv v1 was mapped to ECD2 of human N-cadherin, binding was also observed to ECD1-3-Fc and full length CDH2.

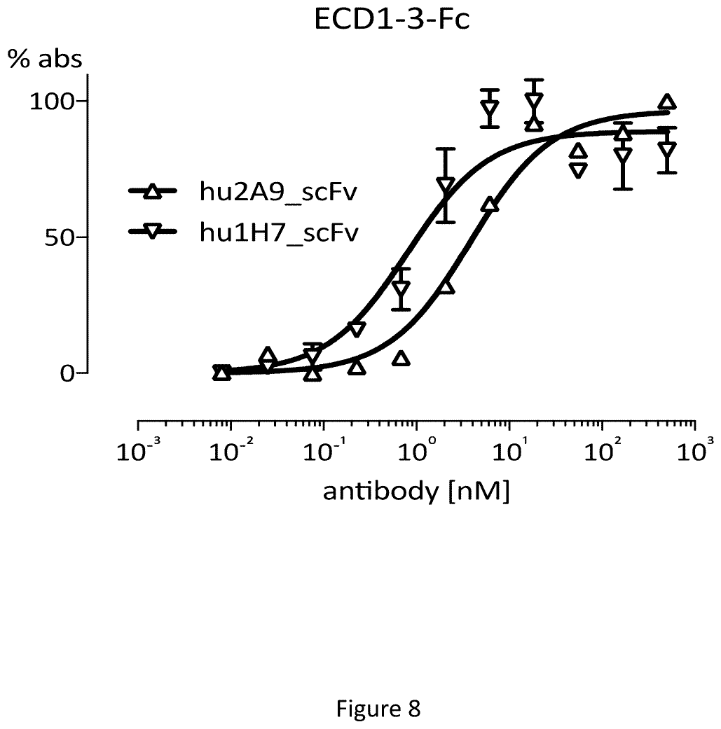

Saturation binding of the humanized scFv fragments was performed on immobilized recombinant human Ncad-ECD1-3-Fc in ELISA (FIG. 8) confirming successful humanization and retained antigen specificity.

hu2A9 IgG and hu1H7 IgG

Full length IgGs were designed based on the humanized variable domains and the constant regions of human Ig gamma 1 heavy chain and human Ig kappa light chain. Gene synthesis, subcloning, transfection of 293 cells and purification of the antibodies from cell supernatant was conducted by GenScript.

Antigen Binding of Humanized Full-Length Antibodies hu2A9 IgG and hu1H7 IgG

Saturation binding of the humanized full-length antibodies in comparison to the parental murine antibodies was tested in ELISA. Binding of hu2A9 IgG to immobilized ECD4 (bacterially produced human N-cadherin ECD4 with ECD3 overhang, used to immunize mice and isolate hybridoma 2A9) was similar to that of mo2A9 IgG with half-maximal binding (Kd) reached at 0.5 nM for hu2A9 IgG and 0.4 nM for mo2A9 IgG. Hybridoma 1H7 was selected against ECD1-3 and binding of the humanized IgG (hu1H7 IgG) to immobilized ECD1-3 (bacterially produced) was comparable (Kd 0.4 nM) to that of the parental murine antibody mo1H7 IgG (Kd 0.3 nM).

### Tumor Growth Inhibition

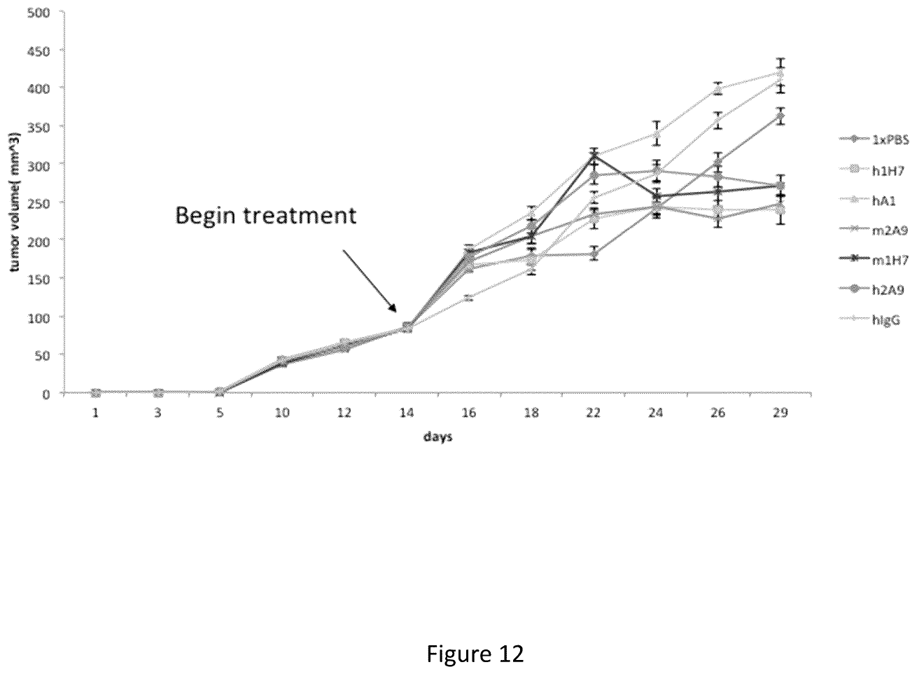

Prostate cancer cell line LNCaP-C1 (N-cadherin-transduced, high expression) shows accelerated in vivo castration-resistant growth compared with low expressing cell lines (LNCaP-C3). LNCaP-C1 cells were implanted subcutaneously into castrated SCID mice and allowed to form tumors (volume 100 mm³). Mice were treated with N-cadherin specific antibodies 3 times per week (10 mg/kg, i.p. administration). Tumor growth in control groups (PBS, isotope human IgG1) was not impaired, while the groups receiving parental mouse antibodies (m2A9, m1H7) or humanized antibodies (h2A9, h1H7) showed inhibited tumor growth (FIG. 12).

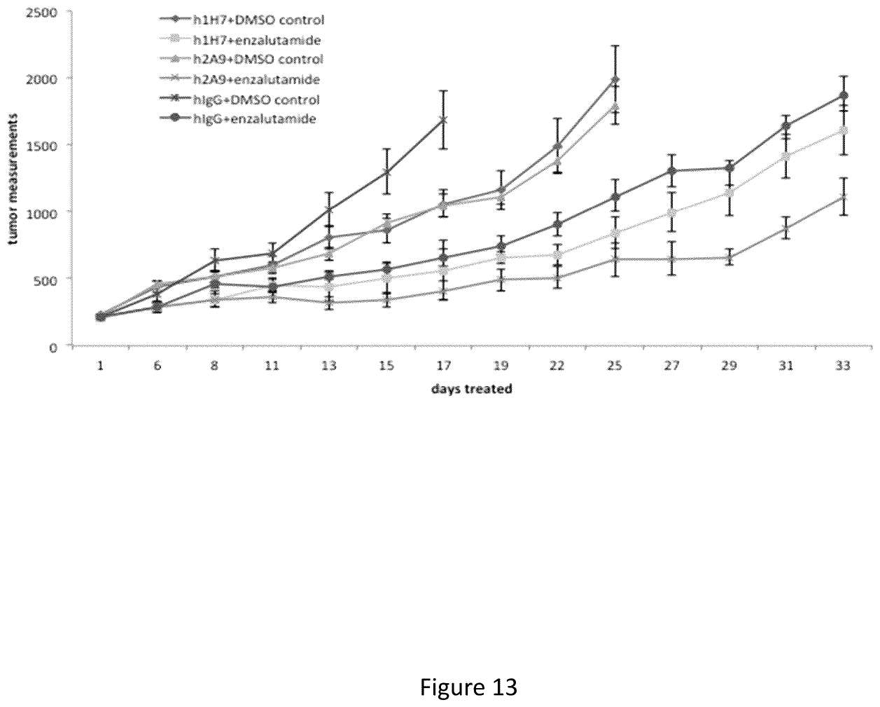

Subcutaneous LNCaP-N (endogenously expressing N-cadherin) xenografts in SCID mice were treated with both anti-N-cadherin humanized antibodies (h2A9 and h1H7) and enzalutamide (synthetic non-steroidal anti-androgen, NSAA) (FIG. 13). Both humanized antibodies inhibited tumor growth compared with the isotype control treatment (hIgG-DMSO control) and had an additive effect when administered together with enzalutamide.

It should be noted that these experiments were performed in SCID mice, absent of a complete immune system and might underestimate the therapeutic activity of the humanized antibodies.

### Example 2: Amino Acid Sequence Information for CDR Regions of a Humanized Anti-N-Cadherin Antibodies of the Present Invention

| | SEQ | | | |
|---|---|---|---|---|
| | ID | | | |
| Antibody | NO | Chain | CDR | Sequence |
| Hu1H7 | 5 | Heavy Chain | CDR1 | GFTFTDYLIQ |
| Hu1H7 | 6 | Heavy Chain | CDR2 | WIGWIYPGSG |
| | | | | SIKYNEKFQG |
| Hu1H7 | 7 | Heavy Chain | CDR3 | ARRGDWGGF |
| | | | | FDY |
| Hu1H7 | 12 | Light Chain | CDR1 | TASSSVSSS |
| | | | | YLHWY |
| Hu1H7 | 13 | Light Chain | CDR2 | LWIFSTSNLAS |
| Hu1H7 | 14 | Light Chain | CDR3 | HQYHRSLT |
| Hu2A9 | 18 | Heavy Chain | CDR1 | GYTFTSYWMQ |
| Hu2A9 | 19 | Heavy Chain | CDR2 | WIGAIYPGDG |
| | | | | ETTYTQKFKG |
| Hu2A9 | 20 | Heavy Chain | CDR3 | AKGDGYWAMDY |
| Hu2A9 | 24 | Light Chain | CDR1 | RSSQSLVHSN |
| | | | | GNTYLHWY |
| Hu2A9 | 25 | Light Chain | CDR2 | LLIYKVSNRFS |
| Hu2A9 | 26 | Light Chain | CDR3 | SQSTHVPLT |

### Example 3: Nucleic Acid Sequence Information for CDR Regions of a Humanized Anti-N-Cadherin Antibodies of the Present Invention

| | SEQ | | | |
|---|---|---|---|---|
| Anti- | ID | | | |
| body | NO | Chain | CDR | Nucleic Sequence |
| Hu1H7 | 27 | Heavy Chain | CDR1 | GGCTTCACCTTCACCGACTA |
| | | | | CCTGATCCAG |
| Hu1H7 | 28 | Heavy Chain | CDR2 | TGGATCGGCTGGATCTACCC |
| | | | | TGGCAGCGGCAGCATCAAGT |
| | | | | ACAACGAGAAGTTCCAGGGC |
| Hu1H7 | 29 | Heavy Chain | CDR3 | GCCAGAAGAGGCGACTGGG |
| | | | | GCGGCTTCTTCGATTAC |
| Hu1H7 | 30 | Light Chain | CDR1 | ACCGCTAGCAGCAGCGTGTC |
| | | | | CAGCAGCTACCTGCACTGGT |
| | | | | AT |
| Hu1H7 | 31 | Light Chain | CDR2 | CTGTGGATCTTCAGCACCAG |
| | | | | CAATCTGGCCTCC |
| Hu1H7 | 32 | Light Chain | CDR3 | CACCAGTACCACAGAAGCCT |
| | | | | GACC |
| Hu2A9 | 33 | Heavy Chain | CDR1 | GGCTACACCTTCACCAGCTA |
| | | | | CTGGATGCAG |
| Hu2A9 | 34 | Heavy Chain | CDR2 | TGGATCGGCGCCATCTATCC |
| | | | | TGGCGACGGCGAGACAACCT |
| | | | | ACACCCAGAAATTCAAGGGC |
| Hu2A9 | 35 | Heavy Chain | CDR3 | GCCAAGGGCGACGGCTACTG |
| | | | | GGCTATGGATTAT |
| Hu2A9 | 36 | Light Chain | CDR1 | CGGAGCAGCCAGAGCCTGGT |
| | | | | GCACAGCAACGGCAACACCT |
| | | | | ACCTGCACTGGTAT |
| Hu2A9 | 37 | Light Chain | CDR2 | CTGCTGATCTACAAGGTGTC |
| | | | | CAACAGATTCAGC |
| Hu2A9 | 38 | Light Chain | CDR3 | TCCCAGAGCACCCACGTGCC |
| | | | | CCTGACC |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A humanized N-cadherin antibody that specifically binds to the extracellular domain of human N-cadherin.

2. The antibody of claim 1, wherein the antibody comprises at least one CDR comprising a sequence that is at least 90% identical to a sequence selected from the group consisting of: VH-CDR1 (SEQ ID NO:5); VH-CDR2 (SEQ ID NO:6); VH-CDR3 (SEQ ID NO:7); VL-CDR1 (SEQ ID NO:12); VL-CDR2 (SEQ ID NO:13); and VL-CDR3 (SEQ ID NO:14).

3. The antibody of claim 1, wherein the antibody comprises the CDRs comprising a sequence that is at least 90% identical to: VH-CDR1 (SEQ ID NO:5); VH-CDR2 (SEQ ID NO:6); VH-CDR3 (SEQ ID NO:7); VL-CDR1 (SEQ ID NO:12); VL-CDR2 (SEQ ID NO:13); and VL-CDR3 (SEQ ID NO:14).

4. The antibody of claim 1, wherein the antibody comprises a heavy chain comprising at least 90% identical to the amino acid sequence of SEQ ID NO: 1.

5. The antibody of claim 1, wherein the antibody comprises a light chain comprising at least 90% identical to the amino acid sequence of SEQ ID NO:8.

6. The antibody of claim 1, wherein the antibody comprises a heavy chain comprising at least 90% identical to the amino acid sequence of SEQ ID NO:1 and a light chain comprising at least 90% identical to the amino acid sequence of SEQ ID NO:8.

7. The antibody of claim 1, wherein the antibody comprises at least one of the CDRs comprising a sequence that is at least 90% identical to a sequence selected from the group consisting of: VH-CDR1 (SEQ ID NO:18); VH-CDR2 (SEQ ID NO:19); VH-CDR3 (SEQ ID NO:20); VL-CDR1 (SEQ ID NO:24); VL-CDR2 (SEQ ID NO:25); and VL-CDR3 (SEQ ID NO:26).

8. The antibody of claim 1, wherein the antibody comprises the CDRs comprising a sequence that is at least 90% identical to: VH-CDR1 (SEQ ID NO:18); VH-CDR2 (SEQ ID NO:19); VH-CDR3 (SEQ ID NO:20); VL-CDR1 (SEQ ID NO:24); VL-CDR2 (SEQ ID NO:25); and VL-CDR3 (SEQ ID NO:26).

9. The antibody of claim 1, wherein the antibody comprises a heavy chain comprising at least 90% identical to the amino acid sequence of SEQ ID NO:15.

10. The antibody of claim 1, wherein the antibody comprises a light chain comprising at least 90% identical to the amino acid sequence of SEQ ID NO:21.

11. The antibody of claim 1, wherein the antibody comprises a heavy chain comprising at least 90% identical to the amino acid sequence of SEQ ID NO:15 and a light chain comprising at least 90% identical to the amino acid sequence of SEQ ID NO:21.

12. The antibody of claim 1, wherein the antibody comprises an effector moiety, wherein the effector moiety is selected from the group consisting of a radioactive label, a fluorescent label, or a therapeutic moiety.

13. A method of treating a N-cadherin-mediated disease or disorder in a subject, comprising the step of administering to said subject an effective amount of any antibody of claims 1-12, wherein when the antibody is administered, the disease or disorder is reduced.

14. The method of claim 13, wherein the disease or disorder is at least selected from the group consisting of prostate cancer, bladder cancer, hormone refractory disease, carcinoma, melanoma, breast cancer, adrenal tumors, or any combinations thereof.

15. The method of claim 13, wherein administration of the anti-N-cadherin antibody inhibits the function of N-cadherin protein.

16. A method of reducing the activity of N-cadherin of a subject, wherein the method comprises administering an effective amount of an antibody to the subject, wherein the antibody comprises any antibody of claims 1-12.

17. A method of detecting an N-cadherin-mediated disease or disorder in a subject, the method comprising administering to the subject any antibody of claims 1-12, detecting the level of the effector moiety, and measuring the level of the effector moiety relative to a comparator control.

18. The method of claim 17 wherein the N-cadherin-mediate disease or disorder is selected from the group consisting of prostate cancer, bladder cancer, hormone refractory disease, or any combinations thereof.

19. A method of reducing the activity of N-cadherin of a subject, wherein the method comprises administering an antibody to the subject wherein the antibody comprises any antibody of claims 1-12.
